(19) United States
(12) Patent Application Publication
(10) Pub. No.: US 2026/0306602 A1
(43) Pub. Date: Oct. 8, 2026

(54) DEVELOPMENT OF CARBOHYDRATE-BASED ANTI-SALMONELLA VACCINES

(71) Applicants: Board of Trustees of Michigan State University, East Lansing, MI (US); UNIVERSITY OF MARYLAND, BALTIMORE, Baltimore, MD (US)

(72) Inventors: Xuefei Huang, Okemos, MI (US); Xingling Pan, East Lansing, MI (US); Chang-Xin Huo, East Lansing, MI (US); Scott Michael Baliban, Washington, DC (US); Sharon Mei Tennant, Ellicott City, MD (US)

(21) Appl. No.: 18/998,478

(22) PCT Filed: Jul. 26, 2023
(86) PCT No.: PCT/US2023/028672

§ 371 Date: Jan. 25, 2025

Related U.S. Application Data
(60) Provisional application No. 63/392,274, filed on Jul. 26, 2022.

(51) Int. Cl. A61K 39/112; A61K 9/00; A61K 39/00; A61K 39/385; A61K 39/39; A61P 31/04

(52) U.S. Cl. CPC A61K 39/0275; A61K 9/0019; A61K 39/385; A61K 39/39; A61P 31/04; A61K 2039/54; A61K 2039/55566; A61K 2039/55583; A61K 2039/575; A61K 2039/6075

(57) ABSTRACT

Provided herein are vaccine compositions comprising Salmonella antigens conjugated to a capsid, wherein said capsid comprises wild type or native sequence, or said capsid may comprise at least one mutation, such as a non-natural mutation; such compositions being useful in the treatment and prevention of a Salmonella infection (salmonellosis), gastroenteritis, typhoid fever, and/or paratyphoid fever; and may be effective against multiple strains of Salmonella.

### CROSS-REFERENCE TO RELATED APPLICATIONS

This application claims the benefit of priority to U.S. Provisional Application Ser. No. 63/392,274, filed on Jul. 26, 2022; the entire contents of said application is incorporated herein in its entirety by this reference.

### BACKGROUND OF THE INVENTION

The emergence of multidrug resistant microbial species suggests that novel prevention and therapeutic strategies are urgently needed. Vaccines are a powerful tool to reduce rates of infection. It is well appreciated that carbohydrates on the surface of microbial cells can be attractive targets for vaccines. However, there are multiple challenges associated with carbohydrate-based vaccine development. The first obstacle lies in the access to microbial associated glycans as carbohydrates from microbial species often have distinctive structures compared to the mammalian counterparts, and can present unique reactivities in glycosylation reactions. The synthesis of these compounds in conjugatable forms needs to be established. Furthermore, carbohydrates alone typically have low immunogenecities, which do not elicit high level and long-lasting antibody responses. Therefore, methods are needed to powerfully boost the immune responses to carbohydrate antigens.

A carrier system is essential to deliver carbohydrate antigens to the immune system and elicit strong anti-carbohydrate antigens antibody responses. However, a potential drawback of protein carrier is that high anti-carrier antibody responses can be induced by the glyco-conjugate. As an example, GD3-KLH generated an-antiGD3 IgG titer of 300, while that for KLH were 1,800,000. The high anti-carrier antibodies can significantly suppress the generation of anti-glycan antibodies. This phenomenon has been reported for carbohydrate based anti-microbial disease vaccines. It has been suggested that an ideal carrier should induce high levels of anti-glycan antibodies without strong anti-self antibodies.

Accordingly, there is a great need in the art to identify potential therapeutic strategies and compositions that activate immune responses in the treatment and prevention of disease and infection. Salmonella infections (salmonellosis) are a major public health problem throughout the world. The development of an effective vaccine is a highly attractive strategy to combat salmonellosis, especially with the rise of multidrug resistant Salmonella strains. Salmonella bacteria bear characteristic glycan structures on the cell surface, which define the specific serovars and at the same time can serve as antigenic targets. Provided herein are strategies and vaccines for targeting the carbohydrates from Salmonella surface, which can be effective against multiple pathogenic strains of Salmonella.

### SUMMARY OF THE INVENTION

Provided herein are vaccine compositions comprising an antigen conjugated to a capsid. In some embodiments, the vaccine composition comprises an antigen conjugated to a wild type capsid. In some embodiments, the vaccine composition comprises an antigen conjugated to a wild type bacteriophage QB capsid. In some embodiments, the vaccine composition comprises an antigen conjugated to a bacteriophage QB capsid having a wild type or native sequence. In some embodiments, the vaccine composition comprises an antigen conjugated to a bacteriophage QB capsid having a wild type or natural sequence set forth in SEQ ID NO: 1. In some embodiments, the vaccine composition comprises an antigen conjugated to a capsid having at least one mutation from the wild type capsid. In some embodiments, the vaccine composition comprises an antigen conjugated to a bacteriophage QB capsid having at least one mutation from the wild type bacteriophage QB capsid. In some embodiments, the at least one mutation comprises a non-natural mutation. In some embodiments, the non-natural mutation comprises a non-natural amino acid mutation.

In certain embodiments of the vaccine composition, the capsid comprises at least one, two, three, four, five, six, seven, eight, nine, ten, eleven, twelve, thirteen, fourteen, fifteen, sixteen, seventeen, eighteen, nineteen, or twenty mutations.

In certain embodiments of the vaccine composition, the at least one non-natural mutation is a disulfide bond mutation.

In some such embodiments of the invention disclosed herein, the antigen conjugated to the capsid is a Salmonella antigen. Such Salmonella antigens may derived from a surface glycan of Salmonella. For example and without limitation, the antigen may be a polysaccharide, such as a tri-, tetra-, penta-, hexa-, hepta-, octa-, non, or dodeca-saccharide. In some preferred embodiments, the Salmonella antigen comprises tri-, hexa-, or nona-saccharides of Salmonella O-polysaccharide backbone. Most preferably, the Salmonella antigen comprises the trisaccharide of Man-Rha-Gal. The vaccine compositions disclosed herein may be used in preventing or treating a Salmonella infection (salmonellosis) gastroenteritis, typhoid fever, and/or paratyphoid fever in a subject in need thereof. Said vaccines may birected to (e.g., provide protection against or treatment for) multiple strains of Salmonella, such as S. enteritidis, S. paratyphi A, S. typhimurum, and S. newport.

### DETAILED DESCRIPTION OF THE INVENTION

Salmonella infections (salmonellosis) are a major public health problem throughout the world. There are multiple pathogenic strains of Salmonella bacteria. Salmonella Typhi (S. typhi) and Salmonella paratyphi A, B and C (S. paratyphi A, B and C) serovars cause enteric fever, while nontyphoidal Salmonella (NTS) serovars generally cause gastroenteritis that can progress to invasive diseases. In Southeast Asia, typhoid and paratyphoid due to S. paratyphi A are endemic. In the US, while typhoid and paratyphoid are rare, there are >1.2 million NTS cases annually, resulting in 10,000-20,000 hospitalizations and hundreds of deaths, for which Salmonella typhimurium (S. typhimurium) and Salmonella enteritidis (S. enteritidis) are most common.

The development of an effective vaccine is a highly attractive strategy to combat salmonellosis, especially with the rise of multidrug resistant Salmonella strains. Salmonella bacteria bear characteristic glycan structures on the cell surface, which define the specific serovars and at the same time can serve as antigenic targets. S. typhi expresses a Vi capsule polysaccharide that covers the bacterial surface and protects the bacterium from the host immune system. S. paratyphi A and most NTS do not express such a capsule, and hence the surface polysaccharide in these serovars is the O polysaccharide of lipopolysaccharide (COPS). Carbohydrate based vaccine constructs have been shown to be effective in protecting the host, an example of which is the ViCPS vaccine targeting the Vi polysaccharide on S. typhi. Although ViCPS offers protection with between 50% to 80% efficacy in the first year, protection wanes after two years. Currently, there are no approved vaccines against any other Salmonella strains. Therefore, there is an urgent need to develop novel anti-Salmonella vaccines.

As noted above, enteric fever, caused by Salmonella paratyphi A and S. typhi, as well as invasive nontyphoidal Salmonella (iNTS) disease, primarily caused by S. enteritidis and S. typhimurium, are responsible for a high burden of disease globally. Multidrug resistance is increasing in prevalence, and there are no licensed vaccines to prevent salmonellosis other than for S. typhi. Vaccines targeting polysaccharides on the surface of pathogenic bacteria have proven to be an effective prevention strategy, as exemplified by S. typhi capsular polysaccharide vaccines. Salmonella serovars paratyphi A, Typhimurium, and Enteritidis are coated with a dense layer of O-polysaccharide that contains a repeating α-D-mannose (Man)-1,4-α-L-rhamnose (Rha)-1,3-α-D-galactose (Gal)-1,2-motif. As disclosed herein, synthetic chemistry and vaccinology were employed to develop carbohydrate-based vaccines against the major invasive species, S. typhimurium, S. enteritidis and S. paratyphi A. The studies disclosed herein explore whether the Man-Rha-Gal motif constitutes a broadly protective antigen by constructing a glycoconjugate vaccine based on this antigen and characterizing its immunogenicity and efficacy against Salmonella infection.

Provided herein is the synthesis of Salmonella-associated carbohydrate antigens. Traditionally, the studies of carbohydrate based anti-Salmonella vaccines have relied on native polysaccharides isolated from the pathogenic strains. The glycans obtained are an inhomogeneous mixture with variable length and structures. Furthermore, there are concerns regarding batch-to-batch variability and potential contaminants. Instead of relying on isolation, synthetic routes were developed to produce structurally well-defined Salmonella associated glycans, including the tetrasaccharide repeating units of O-antigen polysaccharides from S. enteritidis and S. paratyphi A, two pathogenic serovars. As the optimum immunogenic sizes of the glycan antigens are not known, provided herein is synthesis of glycan antigens from tri- and tetra- to dodeca-saccharides from S. enteritidis, S. typhimurum, and S. paratyphi A. In addition, since O polysaccharide of these strains of Salmonella bacteria share a common backbone, glycans were synthesized corresponding to the backbone structures to aid in the development of potential broad-spectrum vaccines.

Also provided herein is the development of Qβ-glycan conjugates as strain specific anti-Salmonella vaccines. One of the major challenges in developing carbohydrate-based vaccines is the poor immunogenecity of the antigens. Previously, it has been demonstrated that virus-like particles exemplified by bacteriophage Qβ are capable of displaying carbohydrate antigens in a highly organized manner. (See WO 2019/045791, incorporated herein by reference in its entirety.) The patterned antigen display led to strong IgG antibody responses against tumor associated carbohydrate antigens. Based on these results, it was hypothesized that Qβ can significantly boost humoral responses against Salmonella associated glycans.

Disclosed herein are strain specific antigenic glycans conjugated with bacteriophage Qβ as potential vaccines. The bioconjugation reactions were optimized, to give high yields of the glyco-conjugates without resorting to a large excess of the valuable carbohydrate substrates. For example, Enteritidis glycan Qβ-conjugate induced very high titers of IgG antibodies (IgG titers over 80,000,000 ELISA units in rabbits) against the glycan. The induced antibodies recognized native O-polysaccharide both as an isolated antigen as well as when present on bacterial cells. Furthermore, transfer of anti-sera from immunized rabbits provided 100% protection to mice against the challenge of a lethal dose of S. enteritidis. Building on these encouraging results, optimum structures of glycan-based antigens were established. Furthermore, novel Qβ carriers were designed to enable the activation of Salmonella-specific helper T cells and the generation of higher levels of anti-glycan IgG antibodies. Effective strain specific anti-Salmonella vaccines for S. enteritis, S. typhimurium as well as S. paratyphi A will be developed.

Further provided herein is the establishment of Qβ-glycan conjugates as broad spectrum anti-Salmonella vaccines. As S. enteritidis, S. typhimurium and S. paratyphi A share a common polysaccharide backbone structure, and differ in its dideoxy hexose branch, the common backbone glycans were targeted for vaccines. Antibodies induced by a single construct made of Qβ conjugated with the backbone glycan can potentially offer broad-spectrum protection against multiple Salmonella strains that would provide an exciting tool for salmonellosis prevention.

To that end, a Man-Rha-Gal oligosaccharide was synthesized and this glycan was conjugated to a bacteriophage Qβ carrier to enhance anti-polysaccharide immunity. New Zealand White rabbits (n=7) were then immunized intramuscularly three times with these conjugates. Vaccination resulted in robust serum IgG responses that recognized the native O-polysaccharides from S. paratyphi A, S. typhimurium, and S. enteritidis in both ELISA (geometric mean titer=6.3×10⁴, 2.1×10⁴, and 3×10⁶ EU/mL, respectively; P<0.05 vs. baseline) and flow cytometry formats. Passive protection studies in naïve CD-1 mice (n=18/group) revealed that immune sera provided excellent protection against systemic challenge with a lethal dose of S. enteritidis R11 (1.3×10⁶ CFU), a bloodstream isolate from Mali (vaccine efficacy=94.4%, P<0.0001 vs. baseline). Altogether, these findings highlight that a single repeating unit of the common Man-Rha-Gal motif is sufficient to induce protective antibody responses.

Aspects of the invention provided herein include a vaccine composition comprising a Salmonella antigen conjugated to a capsid. In some embodiments, said capsid comprises at least one non-natural mutation. In some embodiments, the capsid comprises at least one, two, three, four, five, six, seven, eight, nine, ten, eleven, twelve, thirteen, fourteen, fifteen, sixteen, seventeen, eighteen, nineteen, or twenty mutations. In some such embodiments, at least one non-natural mutation is a disulfide bond mutation.

In some embodiments, the Salmonella antigen is derived from a surface glycan of Salmonella. The Salmonella antigen may be a polysaccharide. For Example and without limitation, the Salmonella antigen comprises tri-, tetra-, penta-, hexa-, hepta-, octa-, non, or dodeca-saccharides. In some preferred embodiments, the Salmonella antigen comprises tri-, hexa-, or nona-saccharides of Salmonella O-polysaccharide backbone. Most preferably the Salmonella antigen comprises at least one trisaccharide of Man-Rha-Gal. For example, the Salmonella antigen may comprise repeats of the Man-Rha-Gal trisaccharide, such as two, three, or more Man-Rha-Gal trisaccharides.

In some embodiments of the invention, the capsid is a bacteriophage capsid. Said bacteriophage may be selected from the group consisting of (a): bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (j) bacteriophage f2; (k) bacteriophage PP7; (1) bacteriophage AP205; and (m) bacteriophage P22. Preferably, the bacteriophage is bacteriophage Qβ. In some such embodiments, the mutation comprises at least one mutation selected from N10K, A38K, A40C, A40S, T75K, D102C, D102S, or A117K, or combination thereof. In some preferred embodiments the mutation comprises A38K. In other embodiments, said capsid comprises at least two mutations selected from A40C/D102C, A40S/D102S, or A43C/Q98C. In further embodiments, said capsid comprises at least three mutations selected from A40C/D102C/K13R or A38K/A40C/D102C.

Embodiments of the invention include methods of preventing or treating a Salmonella infection (salmonellosis) in a subject, the method comprising administering to the subject a vaccine composition disclosed herein. Also provided are methods of preventing or treating gastroenteritis, typhoid fever, and/or paratyphoid fever, the method comprising administering to the subject a vaccine composition provided herein. In some such embodiments, the gastroenteritis is chronic or acute.

The contemplated vaccines compositions may be administered systematically. Such systematic administration may be selected from the group consisting of oral, intravenous, intradermal, intraperitoneal, subcutaneous, and intramuscular administration.

In some embodiments, the vaccines contemplated herein are directed against multiple strains of Salmonella, selected from S. enteritidis, S. paratyphi A, S. typhimurum, and S. newport. In some embodiments, the the Salmonella antigen is derived from a O-polysaccharide backbone present in one or more of S. enteritidis, S. paratyphi A, S. typhimurum, or S. newport.

### BRIEF DESCRIPTION OF FIGURES

FIG. 1 Depicts the cell surface core O-polysaccharides (COPS) of S. paratyphi, S. typhimurium, and S. enteritidis and illustrates the common backbone structure with repeating trisaccharides of α-D-mannose (Man)-1,4-α-L-rhamnose (Rha)-1,3-α-D-galactose (Gal)-1,2.

FIG. 2 illustrates the synthesis schemes for (A) S. enteriditis tetrasaccharide, (B) S. paratyphi A tetrasaccharide, and (C) the structures of penta-saccharide bearing a glucoside on the reducing end Gal, and tetra-saccharide without the paratose unit.

FIG. 3 illustrates the synthesis scheme for S. typhimurium tetrasaccharides.

FIG. 4 illustrates the synthesis scheme for common tri-, hexa- and nona-saccharides of O-polysaccharide backbone.

FIG. 5 illustrates the synthesis scheme for strain-specific Salmonella glycans via conversion of the common tri-, hexa- and nona-saccharides of O-polysaccharide backbones.

FIG. 6 depicts the conjugation of Salmonella associated glycans (SAGs) to bacteriophage carrier.

FIG. 7 depicts high and long-lasting anti-glycan 1 antibody responses induced by Qβ-glycan 1 in mice. Anti-glycan 1 IgG titers induced by Qβ-glycan 1 (1 and 4 μg of glycan) and control group with Qβ only on day 35 after first immunization, as shown in FIG. 7A. Each symbol represents one mouse. Average IgG titers induced by Qβ-glycan 1 (4 μg) over 477 days is shown in FIG. 7B. The persistence trend from the 1 μg group was similar.

FIG. 8 shows ELISA data for IgG antibodies induced by Qβ-glycan 1 against related glycans 2, 17, and 18.

FIG. 9 depicts ELISA analysis of various sera against COPS, showing that anti-sera from rabbits immunized with Qβ-glycan 1 exhibited strong binding to COPS (all sera were diluted 25,000 times. X-axis: Pre-rabbit (pre-immunized sera from rabbits); Rabbit (day 56 sera from rabbits immunized with Qβ-glycan 1); Mouse (day 56 sera from selected two mice immunized with Qβ-glycan 1); mAb (mAb 6347 was against S. paratyphi A glycan; mAb 6391 and 6393 were against the common core of COPS. Each mAb was used at 1:20 dilution.

FIG. 10 shows strong binding of S. enteritidis by antibodies induced by Qβ-glycan 1, and enhanced uptake of bacteria by macrophages. FIG. 10A depicts flow cytometry analysis showing that IgG antibodies in post-immune sera (1:10,000 dilution) from rabbits immunized with Qβ-glycan 1 exhibited significant recognition of S. enteritidis clinical strain R11. FIG. 10B shows that post-immune sera from rabbits immunized with Qβ-glycan 1 enhanced uptake of S. enteritidis bacteria by macrophage J774 cells. Post-immune sera from Qβ immunized or pre-immunized rabbits did not have much impact on macrophage uptake.

FIG. 11 depicts Kaplan-Meier survival curves of mice challenged with lethal doses of S. enteritidis R11. Post immune sera from Qβ-glycan 1 immunized rabbits (at 1:100 or 1:500 dilution) provided complete protection to mice from the challenges (n=12 for each group). In contrast, all mice in the group receiving PBS (n=7) and the group receiving 1:100 dilution of pre-immune sera (n=12) succumbed to bacterial infection by day 8, 11 out of the 12 mice receiving pre-immune sera at 1:500 dilution also died by day 8.

FIG. 12 illustrates reduced anti-carrier antibody titers and significantly improved anti-glycan IgG responses when using rationally designed Qβ mutants. FIG. 12A depicts the calculated relative solvent exposure of amino acid residues of Qβ (1QBE) based on its crystal structure. N-terminal residues K2, K13, and K16 are the sites for glycan conjugation. Residues A38, T75, Q99, and P119 are highly solvent exposed and serve as potential sites for mutation. FIG. 12B shows mutant Qβ/A38K/A40C/D102C conjugated to a representative carbohydrate antigen Tn elicited stronger anti-Tn IgG responses than wildtype (WT) Qβ-Tn. FIG. 12C shows Qβ/A38K/A40C/D102C conjugated to Tn elicited much less antibody against against WT Qβ or Qβ/A38K/A40C/D102C than WT Qβ-Tn (at 409,600 fold serum dilution). The p values were determined through a two-tailed t test.

FIG. 13 depicts encapsidation scheme. FIG. 13A illustrates the RNA-directed encapsidation technique used to package protein inside Qβ. Plasmid 1 contains the sequence for anti-Rev aptamer (aR), coat protein (CP), and hairpin (hp) for binding coat protein. Plasmid 2 contains sequence fo Rev tag and the target protein (TP). Transformation and transcription of the plasmids into E. coli result in the expression of Rev-tagged TP cargo, and bifunctional mRNA. The anti-Rev aptamer in the mRNA binds to the Rev tag and Qβ genome packaging hp binds to the interior of the CP monomers, thus encapsidating the target protein to Qβ interior with the coat protein RNA sequence acting as the linker. FIG. 13B shows Qβ@(RFP) was successfully produced through the RNA encapsidation technique leading to more than 100 fold increase in RFP fluorescence of the capsid.

FIG. 14 depicts anti-COPS IgG ELISA titers in sera from rabbits immunized with the conjugates of WT Qβ with backbone glycans 25, 26, 27 respectively, against COPS from S. paratyphi A, S. enteritidis, S. typhimurum, and S. newport. Significantly higher levels of IgG antibodies were found against COPS from S. paratyphi A, S. enteritidis, and S. typhimurum, while those against the unrelated S. newport were low.

### A. DEFINITIONS

The articles “a” and “an” are used herein to refer to one or to more than one (i.e., to at least one) of the grammatical object of the article. By way of example, “an element” means one element or more than one element.

The term “administering” means providing a pharmaceutical agent or composition to a subject, and includes, but is not limited to, administering by a medical professional and self-administering.

As used herein, “bacteriophage” refers to viruses that infect and replicate within bacterium. In certain embodiments, the bacteriophage is selected from, but not limited to, the group consisting of bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (j) bacteriophage f2; (k) bacteriophage PP7; (1) bacteriophage AP205; and (m) bacteriophage P22. As used herein, “bacteriophage Qβ” (also referred to as “bacteriophage Qb”, “Qβ” and “Qb”) is one of many small RNA bacteriophages infecting Escherichia coli.

As used herein, the “carbohydrate antigen” refers to classes of antigens that elicit elicit strong antibody responses. In certain embodiments, carbohydrate antigens are selected from, but not limited to, Mucins (“MUC1”), Ganglioside GD2 (Ahmed M. et al. FEBS Letters 588:288-297 (2014), Fucosyl GM1, GD2 (including acetylated GD2), GD3 (including acetylated GD3), GM2, Globo-H, Lewis A, Lewis Y, mucins (e.g., MUC1, MUC4), polysialic acid, sialyl-Lewis A, carbohydrates from Salmonella bacteria (or any of the bacterial, virial, or pathogenic diseases provided herein), Tf, Tn, or sTn (Tn, Tf and STn are commonly found on glycoproteins such as MUC1).

The term “inhibit” or “inhibits” means to decrease, suppress, attenuate, diminish, arrest, or stabilize the development or progression of a disease, disorder, or condition, the activity of a biological pathway, or a biological activity, such as the growth of a solid malignancy, e.g., by at least 10%, 20%, 30%, 40%, 50%, 60%, 70%, 80%, 90%, 95%, 98%, 99%, or even 100% compared to an untreated control subject, cell, biological pathway, or biological activity or compared to the target, such as a growth of a solid malignancy, in a subject before the subject is treated. By the term “decrease” is meant to inhibit, suppress, attenuate, diminish, arrest, or stabilize a symptom of a cancer disease, disorder, or condition. It will be appreciated that, although not precluded, treating a disease, disorder or condition does not require that the disease, disorder, condition or symptoms associated therewith be completely eliminated.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically-acceptable salts” refers to the relatively non-toxic, inorganic and organic acid addition salts of compounds.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

A “subject” can include a human subject for medical purposes, such as for the treatment of an existing disease, disorder, condition or the prophylactic treatment for preventing the onset of a disease, disorder, or condition or an animal subject for medical, veterinary purposes, or developmental purposes. Suitable animal subjects include mammals including, but not limited to, primates, e.g., humans, monkeys, apes, gibbons, chimpanzees, orangutans, macaques and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, guinea pigs, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a disease, disorder, or condition. Thus, the terms “subject” and “patient” are used interchangeably herein. Subjects also include animal disease models (e.g., rats or mice used in experiments, and the like).

The term “subject in need thereof” means a subject identified as in need of a therapy or treatment.

The terms “systemic administration,” “administered systemically,” “peripheral administration,” and “administered peripherally” mean the administration of a compound, drug or other material other than directly into the central nervous system, such that it enters the patient's system and, thus, is subject to metabolism and other like processes, for example, subcutaneous administration.

The term “therapeutic agent” or “pharmaceutical agent” refers to an agent capable of having a desired biological effect on a host.

The term “therapeutic effect” refers to a local or systemic effect in animals, particularly mammals, and more particularly humans, caused by a pharmacologically active substance.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The term “treating” a disease in a subject or “treating” a subject having a disease refers to subjecting the subject to a pharmaceutical treatment, e.g., the administration of a vaccine composition as described herein, such that at least one symptom of the disease is decreased, prevented from worsening, or delayed from worsening.

### B. CAPSIDS

Provided herein are vaccine compositions comprising an antigen conjugated to a capsid. In some embodiments, the vaccine composition comprises an antigen conjugated to a wild type capsid. In some embodiments, the vaccine composition comprises an antigen conjugated to a wild type bacteriophage Qβ capsid. In some embodiments, the vaccine composition comprises an antigen conjugated to a bacteriophage Qβ capsid having a wild type or native sequence. In some embodiments, the vaccine composition comprises an antigen conjugated to a bacteriophage Qβ capsid having a wild type or natural sequence set forth in SEQ ID NO: 1. In some embodiments, the vaccine composition comprises an antigen conjugated to a capsid having at least one mutation from the wild type capsid. In some embodiments, the vaccine composition comprises an antigen conjugated to a bacteriophage Qβ capsid having at least one mutation from the wild type bacteriophage Qβ capsid. In some embodiments, the at least one mutation comprises a non-natural mutation. In some embodiments, the non-natural mutation comprises a non-natural amino acid mutation. In some embodiments, the vaccine compositions provided herein comprises an antigen (e.g., carbohydrate antigen, polypeptides, peptides, proteins and small molecules) conjugated to a capsid (e.g., bacteriophage Qb), wherein said capsid comprises at least one mutation (e.g., at least one point mutation, or at least one non-natural disulfide bond). In some embodiments, the capsid are fragments or a portion of the capsid amino acid sequence of sufficient length, that when conjugated to the antigen, can elicit an enhanced and strong immune response. In certain embodiments, the capsid polypeptide also includes amino acids that do not correspond to the naturally occurring capsid amino acid sequence (e.g., comprising at least one point mutation, or at least one non-natural disulfide bond mutation, or a fusion protein comprising a capsid amino acid sequence and an amino acid sequence corresponding to a non-capsid protein or polypeptide).

In some embodiments, the capsid is derived from bacteriophage. In certain embodiments, the bacteriophage is selected from the group consisting of bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (j) bacteriophage f2; (k) bacteriophage PP7; (1) bacteriophage AP205; and (m) bacteriophage P22.

In some embodiments, the capsid has a sequence set forth in coat protein Table A.

TABLE A
Listing of capsid and associated GenBank numbers

| | |
|---|---|
| [Escherichia virus Qbeta] | GenBankAEQ25550.1 |
| coat protein [Enterobacteria phage M11] | GenBank AAC06250.1 |
| coal protein [Enterobacteria phage SP] | GenBank AEQ25562.1 |
| coat protein [Enterobacteria phage FI sensu lato] | Genbank NP_695027.1 |
| capsid protein └Escherichia virus FI┘ | Genbank ACT66758.1 |
| coal protein [Escherichia virus Qbeta] | Genbank ACY07224.1 |
| Chain A, Bacteriophage Q Beta Capsid | Genbank 1QBE_A |
| capsid protein └Escherichia virus Qbeta┘ | ACT66734.1 |
| capsid protein [Escherichia virus Qbeta] | ACT66730.1 |
| capsid protein [Escherichia virus Qbeta] | ACT66742.1 |
| read-through protein [Escherichia virus Qbeta] | ACY07231.1 |
| A1 protein [Escherichia virus Qbeta] | AAA16663.1 |
| read-through protein [Escherichia virus Qbeta] | AEQ25545.1 |
| Minor capsid protein A1 | Q8LTE1.2 |
| read-through protein [Escherichia virus Qbeta] | ACY07227.1 |
| read-through protein [Escherichia virus Qbeta] | ACY07235.1 |
| capsid protein └Escherichia virus Qbeta┘ | ACT66738.1 |
| read-through protein [Escherichia virus Qbeta] | AEQ25549.1 |
| read-through protein [Escherichia virus Qbeta] | AEQ25541.1 |
| read-through protein [Escherichia virus Qbeta] | ACY07223.1 |
| read-through protein [Escherichia virus Qbeta] | ACT66735.1 |
| Chain A, Bacteriophage Qbeta Coat Protein | 4L8H_A |
| In Complex With Rna Operator Hairpin | |
| read-through protein [Escherichia virus Qbeta] | ACT66731.1 |
| read-through protein [Escherichia virus Qbeta] | ACT66743.1 |
| read-through protein [Escherichia virus Qbeta] | ACT66739.1 |
| capsid protein [Escherichia virus Qbeta] | ACT66746.1 |
| major coat protein [Escherichia virus Qbeta] | NP_046751.1 |
| read-through protein [Escherichia virus Qbeta] | ACT66747.1 |
| minor coat protein [Escherichia virus Qbeta] | NP_046750.1 |
| coat protein [Enterobacteria phage SP] | ACY07244.1 |
| coat protein [Enterobacteria phage SP] | ACY07240.1 |
| Al-protein [Enterobacteria phage M11] | AAC06251.1 |
| coat protein [Enterobacteria phage SP] | AEQ25558.1 |
| capsid protein [Escherichia virus FI] | ACT66762.1 |
| major coat protein [Escherichia virus FI] | YP_009208147.1 |
| capsid protein [Escherichia virus FI] | ACT66750.1 |
| coat protein [Enterobacteria phage SP] | ACY07248.1 |
| read-through protein [Enterobacteria phage SP] | ACY07251.1 |
| read-through protein [Enterobacteria phage SP] | AEQ25561.1 |
| read-through protein [Enterobacteria phage SP] | ACY07243.1 |
| Minor capsid protein A1 | P09677.1 |
| readthrough protein [Enterobacteria phage FI sensu lato] | NP_695026.1 |
| capsid protein [Escherichia virus FI] | ACT66754.1 |
| read-through protein [Enterobacteria phage SP] | AEQ25553.1 |
| read-through protein [Enterobacteria phage SP] | ACY07239.1 |
| read-through protein [Enterobacteria phage SP] | AEQ25557.1 |
| read-through protein [Escherichia virus FI] | ACT66759.1 |
| major coat protein [Enterobacteria phage NL95] | AAC14703.1 |
| read-through protein [Escherichia virus FI] | ACT66763.1 |
| read-through protein [Enterobacteria phage SP] | ACY07247.1 |
| A1-protein [Escherichia virus FI] | YP_009208146.1 |
| read-through protein [Escherichia virus FI] | ACT66751.1 |
| read-through protein [Escherichia virus FI] | ACT66755.1 |
| A1-protein [Enterobacteria phage NL95] | AAC14704.1 |

In some embodiments, the bacteriophage Qβ capsid comprises a sequence set forth in SEQ ID NO: 1.

(SEQ ID NO: 1)
(M)AKLETVTLGNIGKDGKQTLVLNPRGVNPTNGVASLSQAGAVPALEK
RVTVSVSQPSRNRKNYKVQVKIQNPTACTANGSCDPSVTRQAYADVTFS
FTQYSTDEERAFVRTELAALLASPLLIDAIDQLNPAY

In some embodiments, the bacteriophage Qβ capsid comprises at least one mutation set forth in Table B.

TABLE B
Qβ mutants reported that assemble to form the capsid.

| Entry | Mutation(s) | category | Yield(mg/L) | Tm (° C.) |
|---|---|---|---|---|
| 1 | WT | − | ++ | 83.3 |
| 2^{42a} | K16M | exterior charge/ | na | na |
| | | reactive site | | |
| 3^{42a} | T93M | internal | na | na |
| | | reactive site | | |
| 3^{42b} | D14R | exterior charge | − | na |
| 4^{42b} | N10R | exterior charge | ++ | na |
| 5^{42b} | T18R | exterior charge | ++ | na |
| 6^{42b} | D14R/T18R | exterior charge | − | unstable |
| 7^{7a} | C74S | Remove disulfide | + | 61.7 |
| 8^{7a} | C74S/C80S | Remove disulfide | ++ | 61 |
| 9^{7a} | | Interdimer | | |
| 10^{7a} | | Interdimer | | |
| | | salt bridge | | |
| 11^{7a} | Q65H | RNA binding | ++ | 78.6 |
| 12^{7a} | D91N | RNA binding | + | 79.4 |
| 13^{7a} | Q65H/D91N | RNA binding | + | 81.6 |
| 14^{7a} | Y62F/C74S/C80S | combination | ++ | 60.4 |
| 15^{7a} | D81N/C74S/C80S | combination | ++ | 62 |
| 16^{7a} | Q65H/C74S | combination | + | 60.7 |
| 17^{7a} | D91N/C74S | combination | + | 62.1 |
| 18^{7a} | Q65H/D91N/C74S | combination | ++ | 61.4 |
| 19^{7a} | | interdimer | | |
| | | H-bond | | |
| 20^{7a} | Y62W | Trp replace | + | 77.0 |
| 21^{7a} | Y99W | Trp replace | + | 74.8 |
| 22^{7a} | Y132W | C-term. | + | 80.9 |
| | | Trp replace | | |
| 23^{7a} | L35W | Trp replace | ++ | 72.2 |
| 24^{7a} | P23A | structure | − | 73.8 |
| 25^{7a} | K2Q | exterior charge | ++ | 74.5 |
| 26^{7a} | K13Q/K16Q | exterior charge | ++ | 75.6 |
| 27^{7a} | A1S | conjugation | + | 73.9 |
| | | handle | | |
| 28⁴³ | K13E | exterior charge | na | na |
| 29⁴³ | K13Q | exterior charge | na | na |
| 30⁴³ | K16E | exterior charge | na | na |
| 31⁴³ | K16Q | exterior charge | na | na |
| 32⁴³ | K16F | exterior charge | na | na |
| 33⁴³ | K16Y | exterior charge | na | na |
| 34⁴³ | K46Q | exterior charge | na | na |
| 35⁴³ | K13Q/K16Q | exterior charge | na | na |

Note:
Yield ++: >80 mg/L, +: >20-80 mg/L, −: <20 mg/L, na: not applicable
^{7a}corresponds to Fiedler, J et al. Biomacromolecules 2012, 13 (8), 2339-2348;
^{42a}corresponds to Prasuhn, D et al. JACS 2008, 130 (4), 1328-1334;
^{42b}corresponds to Udit, A et al. ChemBioChem 2009, 10 (3), 503-510.
⁴³corresponds to Hovlid, M. L et al. The Scripps Research Institute, La Jolla, 2014.

In certain embodiments, the capsid comprises at least two, three, four, five, six, seven, eight, nine, ten, eleven, twelve, thirteen, fourteen, fifteen, sixteen, seventeen, eighteen, nineteen, or twenty mutations. In some embodiments, the mutation is a non-natural disulfide bond mutation. In some embodiments, the capsid comprises, but not limited to, the following mutations (Table C).

TABLE C
Listing of bacteriophage Qß capsid

N10K (Asparagine at position 10 of SEQ ID NO: 1 mutated to Lysine)
K13R (Lysine at position 13 of SEQ ID NO: 1 mutated to Arginine)
A38K (Alanine at position 38 of SEQ ID NO: 1 mutated to Lysine)
A40C (Alanine at position 40 of SEQ ID NO: 1 mutated to Cysteine)
A40S (Alanine at position 40 of SEQ ID NO: 1 mutated to Serine)
T75K (Threonine at position 75 of SEQ ID NO: 1 mutated to Lysine)
D102C (Aspartic acid at position 102 of SEQ ID NO: 1 mutated to Cysteine)
D102S (Aspartic acid at position 102 of SEQ ID NO: 1 mutated to Serine)
A117K (Alanine at position 117 of SEQ ID NO: 1 mutated to Lysine)
A40C/D102C (double mutant wherein the Alanine at position 40 of SEQ ID NO: 1 mutated
to Cysteine and the Aspartic acid at position 102 of SEQ ID NO: 1 mutated to Cysteine)
A40S/D102S (double mutant wherein the Alanine at position 40 of SEQ ID NO: 1 mutated
to Serine and the Aspartic acid at position 102 of SEQ ID NO: 1 mutated to Serine).
A43C/N98C (double mutant wherein the Alanine at position 43 of SEQ ID NO: 1 mutated
to Cysteine and the Glutamine at position 98 of SEQ ID NO: 1 mutated to Cysteine).
A40C/D102C/K13R (triple mutant wherein the Alanine at position 40 of SEQ ID NO: 1
mutated to Cysteine, the Aspartic acid at position 102 of SEQ ID NO: 1 mutated to
Cysteine, and the Lysine at position 13 of SEQ ID NO: 1 mutated to Arginine)
A38K/A40C/D102C (triple mutant wherein Alanine at position 38 of SEQ ID NO: 1
mutated to Lysine, the Alanine at position 40 of SEQ ID NO: 1 mutated to Cysteine, and
the Aspartic acid at position 102 of SEQ ID NO: 1 mutated to Cysteine).
Additional single point mutations may comprise, but not limited to, point mutations at K2,
L4, V7, N10, K13, D14, K16, Q18, L20, A38, G40, E46, V67, T75, V85, Q99, E103,
A117, P119, L122, or D127 of SEQ ID NO: 1, or combinations thereof.
N10K
(M)AKLETVTLGKIGKDGKQTLVLNPRGVNPTNGVASLSQAGAVPALEKRVTVSVS
QPSRNRKNYKVQVKIQNPTACTANGSCDPSVTRQAYADVTFSFTQYSTDEERAFV
RTELAALLASPLLIDAIDQLNPAY (SEQ ID NO: 2)
A38K
(M)AKLETVTLGNIGKDGKQTLVLNPRGVNPTNGVASLSQKGAVPALEKRVTVSVS
QPSRNRKNYKVQVKIQNPTACTANGSCDPSVTRQAYADVTFSFTQYSTDEERAFV
RTELAALLASPLLIDAIDQLNPAY (SEQ ID NO: 4)
A40C
(M)AKLETVTLGNIGKDGKQTLVLNPRGVNPTNGVASLSQAGCVPALEKRVTVSVS
QPSRNRKNYKVQVKIQNPTACTANGSCDPSVTRQAYADVTFSFTQYSTDEERAFV
RTELAALLASPLLIDAIDQLNPAY (SEQ ID NO: 5)
A40S
(M)AKLETVTLGNIGKDGKQTLVLNPRGVNPTNGVASLSQAGSVPALEKRVTVSVS
QPSRNRKNYKVQVKIQNPTACTANGSCDPSVTRQAYADVTFSFTQYSTDEERAFV
RTELAALLASPLLIDAIDQLNPAY (SEQ ID NO: 6)
T75K
(M)AKLETVTLGNIGKDGKQTLVLNPRGVNPTNGVASLSQAGAVPALEKRVTVSVS
QPSRNRKNYKVQVKIQNPTACKANGSCDPSVTRQAYADVTFSFTQYSTDEERAFV
RTELAALLASPLLIDAIDQLNPAY (SEQ ID NO: 7)
D102C
(M)AKLETVTLGNIGKDGKQTLVLNPRGVNPTNGVASLSQAGAVPALEKRVTVSVS
QPSRNRKNYKVQVKIQNPTACTANGSCDPSVTRQAYADVTFSFTQYSTCEERAFV
RTELAALLASPLLIDAIDQLNPAY (SEQ ID NO: 8)
D102S
(M)AKLETVTLGNIGKDGKQTLVLNPRGVNPTNGVASLSQAGAVPALEKRVTVSVS
QPSRNRKNYKVQVKIQNPTACTANGSCDPSVTRQAYADVTFSFTQYSTSEERAFVR
TELAALLASPLLIDAIDQLNPAY (SEQ ID NO: 9)
A117K
(M)AKLETVTLGNIGKDGKQTLVLNPRGVNPTNGVASLSQAGAVPALEKRVTVSVS
QPSRNRKNYKVQVKIQNPTACTANGSCDPSVTRQAYADVTFSFTQYSTDEERAFV
RTELAALLKSPLLIDAIDQLNPAY (SEQ ID NO: 10)
A40C/D102C
(M)AKLETVTLGNIGKDGKQTLVLNPRGVNPTNGVASLSQAGCVPALEKRVTVSVS
QPSRNRKNYKVQVKIQNPTACTANGSCDPSVTRQAYADVTFSFTQYSTCEERAFV
RTELAALLASPLLIDAIDQLNPAY (SEQ ID NO: 11)
A40S/D102S
(M)AKLETVTLGNIGKDGKQTLVLNPRGVNPTNGVASLSQAGCVPALEKRVTVSVS
QPSRNRKNYKVQVKIQNPTACTANGSCDPSVTRQAYADVTFSFTQYSTSEERAFVR
TELAALLASPLLIDAIDQLNPAY (SEQ ID NO: 12)
A40C/D102C/K13R
(M)AKLETVTLGNIGRDGKQTLVLNPRGVNPTNGVASLSQAGCVPALEKRVTVSVS
QPSRNRKNYKVQVKIQNPTACTANGSCDPSVTRQAYADVTFSFTQYSTCEERAFV
RTELAALLASPLLIDAIDQLNPAY (SEQ ID NO: 13)
A43C/Q98C
(M)AKLETVTLGNIGKDGKQTLVLNPRGVNPTNGVASLSQAGAVPCLEKRVTVSVS
QPSRNRKNYKVQVKIQNPTACTANGSCDPSVTRQAYADVTFSFTCYSTDEERAFV
RTELAALLASPLLIDAIDQLNPAY (SEQ ID NO: 14)
A38K/A40C/D102C
(M)AKLETVTLGNIGKDGKQTLVLNPRGVNPTNGVASLSQKGCVPALEKRVTVSVS
QPSRNRKNYKVQVKIQNPTACTANGSCDPSVTRQAYADVTFSFTQYSTCEERAFV
RTELAALLASPLLIDAIDQLNPAY (SEQ ID NO: 15)

In certain embodiments, bacteriophage Qb mutants may possess the following physical characteristics.

TABLE D
Physical characteristics of Qβ mutants.

| | | SEC | Z-Ave | | Zeta |
|---|---|---|---|---|---|
| Qβ Mutants | Yield(mg/L) | rv. (mL) | (d · nm) | PDI | potential |
| WT | 60 | 11.7 | 28.70 | 0.046 | −2.89 |
| T7K | 10 | 11.8 | 29.00 | 0.278 | −1.08 |
| N10K | 12 | 11.7 | 28.72 | 0.192 | −1.90 |
| A38K | 16 | 12.7 | 26.58 | 0.083 | −1.43 |
| T75K | 25 | 12.0 | 29.02 | 0.096 | −1.64 |
| A117K | 31 | 12.1 | 29.73 | 0.187 | −1.54 |
| P119K | 22 | 12.1 | 29.72 | 0.070 | −2.00 |
| A40C/D102C | 38 | 11.8 | 29.06 | 0.031 | −1.67 |
| A38K/ | 26 | 12.6 | 27.55 | 0.028 | −1.55 |
| A40C/D102C | | | | | |
| A40S/D102S | 20 | 12.0 | 27.82 | 0.028 | −1.37 |
| A40C/ | 15 | 11.9 | 28.86 | 0.119 | −1.97 |
| D102C/K13R | | | | | |

In some embodiments, the bacteriophage Qb capsid comprises a polypeptide comprising amino acid sequences at least 50%, 55%, 60%, 65%, 70%, 75%, 80%, 85%, 90%, 91%, 92%, 93%, 94%, 95%, 96%, 97%, 98% or 99% identical to the amino acid sequence set forth in SEQ ID NOs: 1-15. In some embodiments of the methods, compositions, and kits provided herein, the Qb mutant has at least one mutation set forth in Table C. In some embodiments, the bacteriophage Qβ capsid comprises a wild type or native sequence. In some embodiments, the bacteriophage Qβ capsid comprises a sequence consisting essentially of the sequence set forth in SEQ ID NO: 1. In some embodiments, the Qb mutant has an amino acid sequence that consists essentially of the mutations set forth in SEQ ID NOs: 2-15. In some embodiments, the capsid comprises a polypeptide comprising amino acid sequences at least 50%, 55%, 60%, 65%, 70%, 75%, 80%, 85%, 90%, 91%, 92%, 93%, 94%, 95%, 96%, 97%, 98% or 99% identical to the amino acid sequence set forth in the GenBank numbers set forth in Table A.

In some embodiments of the methods, compositions and kits provided herein, the vaccine composition comprises a capsid having an amino acid sequence that consists of 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130 of SEQ ID Nos: 1-15, or biologically active variant thereof, or combinations thereof, or consecutive amino acids that are at least 70%, 75%, 80%, 85%, 90%, 91%, 92%, 93%, 94%, 95%, 96%, 97%, 98% or 99% identical to an amino acid sequence for a capsid (e.g., SEQ ID NOs: 1-15, or any of the GenBank numbers set forth in Table A). In some embodiments, the bacteriophage Qβ capsid comprises a wild type or native sequence. In some embodiments, the bacteriophage Qβ capsid comprises a sequence consisting essentially of the sequence set forth in SEQ ID NO: 1.

As is well-known to those skilled in the art, polypeptides having substantial sequence similarities can cause identical or very similar immune reaction in a host animal. Accordingly, in some embodiments, a derivative, equivalent, variant, fragment, or mutant of the Qb capsid, or fragment thereof, can also suitable for the methods, compositions and kits provided herein.

In some embodiments, the altered polypeptide may have an altered amino acid sequence, for example by conservative substitution, yet still elicits an enhanced immune response, and are considered functional equivalents. As used herein, the term “conservative substitution” denotes the replacement of an amino acid residue by another, biologically similar residue. It is well known in the art that the amino acids within the same conservative group can typically substitute for one another without substantially affecting the function of a protein. According to certain embodiments, the derivative, equivalents, variants, or mutants of the Qb are at least 85% homologous to a sequence set forth in SEQ ID NOs: 1-15, or biologically active variant thereof, or combinations thereof. In some embodiments, the homology is at least 90%, at least 91%, at least 92%, at least 93%, at least 94%, at least 95%, at least 96%, at least 97%, at least 98%, or at least 99%. In some embodiments, the bacteriophage Qβ capsid comprises a wild type or native sequence. In some embodiments, the bacteriophage Qβ capsid comprises a sequence consisting essentially of the sequence set forth in SEQ ID NO: 1.

### C. VACCINE COMPOSITIONS AND PHARMACEUTICAL COMPOSITIONS/FORMULATIONS OF SAME

Provided herein are vaccine compositions comprising an antigen conjugated to a capsid. In some embodiments, the vaccine composition comprises an antigen conjugated to a wild type capsid. In some embodiments, the vaccine composition comprises an antigen conjugated to a wild type bacteriophage Qβ capsid. In some embodiments, the vaccine composition comprises an antigen conjugated to a bacteriophage Qβ capsid having a wild type or native sequence. In some embodiments, the vaccine composition comprises an antigen conjugated to a bacteriophage Qβ capsid having a wild type or natural sequence set forth in SEQ ID NO: 1. In some embodiments, the vaccine composition comprises an antigen conjugated to a capsid having at least one mutation from the wild type capsid. In some embodiments, the vaccine composition comprises an antigen conjugated to a bacteriophage Qβ capsid having at least one mutation from the wild type bacteriophage Qβ capsid. In some embodiments, the at least one mutation comprises a non-natural mutation. In some embodiments, the non-natural mutation comprises a non-natural amino acid mutation. In some embodiments, the vaccine compositions provided herein comprises an antigen (e.g., carbohydrate antigen, polypeptides, peptides, proteins, and small molecules) conjugated to a capsid (e.g., bacteriophage Qb), wherein said capsid comprises at least one mutation (e.g., at least one point mutation, at least one non-natural amino acid mutation, or at least one non-natural disulfide bond mutation). The capsid is described in Section B, supra. In some embodiments, the antigens are fragments or a portion of the carbohydrate antigen of sufficient length, that when conjugated to the capsid, can elicit an enhanced and strong immune response. The capsid may be conjugated to a plurality of multiple antigens that are the same or different antigen. In certain embodiments, the antigen is a protein and peptide is selected from, but not limited to, TNFalpha, IL1α, IL1β, tau protein, PCSK9, or amyloid B. In some embodiments, the antigen is a small molecule selected from, but not limited to, nicotine, cocaine, or advanced glycation product.

In some embodiments, the present invention provides pharmaceutically acceptable compositions which comprise a therapeutically-effective amount of one or more vaccine compositions (e.g., one or more Qb wild type, or Qβ mutant, antigen conjugate as described above), formulated together with one or more pharmaceutically acceptable carriers (additives) and/or diluents. In another aspect, the compositions can be administered as such or in admixtures with pharmaceutically acceptable carriers and can also be administered in conjunction with other therapies. Conjunctive therapy thus includes sequential, simultaneous and separate, or co-administration of the composition, wherein the therapeutic effects of the first administered has not entirely disappeared when the subsequent compound is administered. In some embodiments, at least one vaccine compositions (e.g., one or more Qβ wild type, or Qβ mutant, antigen conjugate as described above) may be provided to the subject alone or in combination with at least one therapeutic drug, chemotherapeutic agents, scavenger compounds, antibiotics, anti-virals, anti-fungals, anti-inflammatories, vasoconstrictors and anticoagulants, antigens useful for vaccine applications or corresponding pro-drugs.

As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

As set out above, certain embodiments of the one or more vaccine compositions (e.g., one or more Qβ wild type, or Qβ mutant, antigen conjugate as described above) may contain a basic functional group, such as amino or alkylamino, and are, thus, capable of forming pharmaceutically-acceptable salts with pharmaceutically-acceptable acids. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more vaccine compositions (e.g., one or more Qβ wild type, or Qβ mutant, antigen conjugate as described above) may contain one or more acidic functional groups and, thus, are capable of forming pharmaceutically-acceptable salts with pharmaceutically-acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The one or more vaccine compositions (e.g., one or more Qβ wild type, or Qβ mutant, antigen conjugate as described above) may be formulations suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, a formulation of one or more vaccine compositions (e.g., one or more Qβ wild type, or Qβ mutant, antigen conjugate as described above) can comprise other carriers to allow more stability, to allow more stability, different releasing properties in vivo, targeting to a specific site, or any other desired characteristic that will allow more effective delivery of the one or more vaccine compositions (e.g., one or more Qβ wild type, or Qβ mutant, antigen conjugate as described above) to a subject or a target in a subject, such as, without limitation, liposomes, microspheres, nanospheres, nanoparticles, bubbles, micelle forming agents, e.g., bile acids, and polymeric carriers, e.g., polyesters and polyanhydrides. In certain embodiments, an aforementioned formulation renders orally bioavailable a compound of the present invention.

Liquid dosage formulations of one or more vaccine compositions (e.g., one or more Qβ wild type, or Qβ mutant, antigen conjugate as described above) include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an active ingredient. One or more vaccine compositions (e.g., one or more Qβ wild type, or Qβ mutant, antigen conjugate as described above) may also be administered as a bolus, electuary or paste.

In solid dosage forms (e.g., capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. Compositions may also be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Formulations for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate.

Dosage forms for the topical or transdermal administration of one or more vaccine compositions (e.g., one or more Qβ wild type, or Qβ mutant, antigen conjugate as described above) include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions suitable for parenteral administration can comprise sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

In certain embodiments, the above-described pharmaceutical compositions can be combined with other pharmacologically active compounds (“second active agents”) known in the art according to the methods and compositions provided herein. Second active agents can be large molecules (e.g., proteins) or small molecules (e.g., synthetic inorganic, organometallic, or organic molecules). In one embodiment, second active agents independently or synergistically help to treat cancer.

In another embodiment, the composition of the invention may comprise other biologically active substances, including therapeutic drugs or pro-drugs, for example, other chemotherapeutic agents, scavenger compounds, antibiotics, anti-virals, anti-fungals, anti-inflammatories, vasoconstrictors and anticoagulants, antigens useful for cancer vaccine applications or corresponding pro-drugs.

Exemplary scavenger compounds include, but are not limited to thiol-containing compounds such as glutathione, thiourea, and cysteine; alcohols such as mannitol, substituted phenols; quinones, substituted phenols, aryl amines and nitro compounds.

Various forms of the chemotherapeutic agents and/or other biologically active agents may be used. These include, without limitation, such forms as uncharged molecules, molecular complexes, salts, ethers, esters, amides, and the like, which are biologically active.

### D. THERAPEUTIC METHODS

Provided herein are vaccine compositions for the treatment of and/or prevention of diseases and conditions for which an enhanced immune response may be beneficial. Such diseases include, but not limited to, pathogenic infections (e.g., bacterial, viral, or fungal infections) and cancer. In some embodiments, the vaccine compositions described herein may be use as an immunotherapy.

In some embodiments, the vaccine compositions (e.g., one or more Qβ wild type, or Qβ mutant, antigen conjugate as described above) are useful in the treatment of diseases including, but not limited to, persistent infectious disease, sexually transmitted diseases, gastro-intestinal diseases, pulmonary diseases, cardiovascular diseases, stress- and fatigue-related disorders, fungal diseases, pathogenic diseases, viral infections, or bacterial infections.

Viral infectious diseases including human papilloma virus (HPV), hepatitis A Virus (HAV), hepatitis B Virus (HBV), hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, influenza virus, Hepatitis A and B, FIV, lentiviruses, pestiviruses, West Nile Virus, measles, smallpox, cowpox, ebola, coronavirus, retrovirus, herpesvirus, potato S virus, simian Virus 40 (SV40), Mouse Mammary Tumor Virus (MMTV) promoter, Moloney virus, ALV, Cytomegalovirus (CMV), Epstein Barr Virus (EBV), Zika, or Rous Sarcoma Virus (RSV).

In addition, bacterial, fungal and other pathogenic diseases are included, such as Aspergillus, Brugia, Candida, Chikungunya, Chlamydia, Coccidia, Cryptococcus, Dengue, Dirofilaria, Gonococcus, Histoplasma, Leishmania, Mycobacterium, Mycoplasma, Paramecium, Pertussis, Plasmodium, Pneumococcus, Pneumocystis, P. vivax in Anopheles mosquito vectors, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Clostridium tetani, Clostridium botulinum; or, a fungus, such as, e.g., Paracoccidioides brasiliensis; or other pathogen, e.g., Plasmodium falciparum.

Also included are National Institute of Allergy and Infectious Diseases (NIAID) priority pathogens. These include Category A agents, such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B agents, such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain O157: H7, Vibrio cholerae, Cryptosporidium parvum; Category C agents, such as nipah virus, hantaviruses, yellow fever in Aedes mosquitoes, and multidrug-resistant tuberculosis; helminths, such as Schistosoma and Taenia; and protozoa, such as Leishmania (e.g., L. mexicana) in sand flies, Plasmodium, Chagas disease in assassin bugs.

Bacterial pathogens include, but are not limited to, such as bacterial pathogenic gram-positive cocci, which include but are not limited to: pneumococci; staphylococci; and streptococci. Pathogenic gram-negative cocci include: meningococci; and gonococci. Pathogenic enteric gram-negative bacilli include: enterobacteriaceae; pseudomonas, acinetobacteria and eikenella; melioidosis; Salmonella; shigellosis; hemophilus; chancroid; brucellosis; tularemia; Yersinia (pasteurella); streptobacillus moniliformis and spirilum; Listeria monocytogenes; erysipelothrix rhusiopathiae; diphtheria; cholera; anthrax; and donovanosis (Granuloma inguinale). Pathogenic anaerobic bacteria include; tetanus; botulism; other clostridia; tuberculosis; leprosy; and other mycobacteria. Pathogenic spirochetal diseases include: syphilis; treponematoses: yaws, pinta and endemic syphilis; and leptospirosis. Other infections caused by higher pathogen bacteria and pathogenic fungi include: actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include rickettsial and rickettsioses. Examples of mycoplasma and chlamydial infections include: Mycoplasma pneumoniae; lymphogranuloma venereum; psittacosis; and perinatal chlamydial infections. Pathogenic protozoans and helminths and infections eukaryotes thereby include: amebiasis; malaria; leishmaniasis; trypanosomiasis; toxoplasmosis; Pneumocystis carinii; giardiasis; trichinosis; filariasis; schistosomiasis; nematodes; trematodes or flukes; and cestode (tapeworm) infections.

In one embodiment, a method of treatment comprises administering to a subject (e.g., a subject in need thereof), an effective amount of one or more vaccine compositions (e.g., one or more Qβ wild type, or Qβ mutant, antigen conjugate as described above). A subject in need thereof may include, for example, a subject who has been diagnosed with a tumor, including a pre-cancerous tumor, a cancer, or a subject who has been treated, including subjects that have been refractory to the previous treatment.

The term “effective amount,” as in “a therapeutically effective amount,” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

The compositions described herein may be delivered by any suitable route of administration, including orally, nasally, transmucosally, ocularly, rectally, intravaginally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments or drops (including eyedrops), including buccally and sublingually, transdermally, through an inhalation spray, or other modes of delivery known in the art.

The terms “systemic administration,” “administered systemically,” “peripheral administration,” and “administered peripherally” as used herein mean the administration of the vaccine compositions described herein such that it enters the patient's system and, thus, is subject to metabolism and other like processes.

The terms “parenteral administration” and “administered parenterally” as used herein mean modes of administration other than enteral and topical administration, usually by injection, and includes, without limitation, intravenous, intramuscular, intarterial, intrathecal, intracapsular, intraorbital, intraocular, intracardiac, intradermal, intraperitoneal, transtracheal, subcutaneous, subcuticular, intraarticular, subcapsular, subarachnoid, intraspinal and intrasternal injection and infusion.

In certain embodiments the pharmaceutical compositions are delivered generally (e.g., via oral or parenteral administration). In certain other embodiments the pharmaceutical compositions are delivered locally through direct injection into a tumor or direct injection into the tumor's blood supply (e.g., arterial or venous blood supply). In some embodiments, the pharmaceutical compositions are delivered by both a general and a local administration. For example, a subject with a tumor may be treated through direct injection of a composition containing a composition described herein into the tumor or the tumor's blood supply in combination with oral administration of a pharmaceutical composition of the present invention. If both local and general administration is used, local administration can occur before, concurrently with and/or after general administration.

In some embodiments, the subject pharmaceutical compositions of the present invention will incorporate the substance or substances to be delivered in an amount sufficient to deliver to a patient a therapeutically effective amount of an incorporated therapeutic agent or other material as part of a prophylactic or therapeutic treatment. The desired concentration of the active compound in the particle will depend on absorption, inactivation, and excretion rates of the drug as well as the delivery rate of the compound. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

Dosage may be based on the amount of the one or more vaccine compositions (e.g., one or more Qβ wild type, or Qβ mutant, antigen conjugate as described above) per kg body weight of the patient. For example, a range of amounts of compositions or compound encapsulated therein are contemplated, including about 0.001, 0.01, 0.1, 0.5, 1, 10, 15, 20, 25, 50, 75, 100, 150, 200 or 250 mg or more of such compositions per kg body weight of the patient. Other amounts will be known to those of skill in the art and readily determined.

In certain embodiments, the dosage of the one or more vaccine compositions (e.g., one or more Qβ wild type, or Qβ mutant, antigen conjugate as described above) will generally be in the range of about 0.001 mg to about 250 mg per kg body weight, specifically in the range of about 50 mg to about 200 mg per kg, and more specifically in the range of about 100 mg to about 200 mg per kg. In one embodiment, the dosage is in the range of about 150 mg to about 250 mg per kg. In another embodiment, the dosage is about 200 mg per kg.

In some embodiments the molar concentration of the one or more vaccine compositions (e.g., one or more Qβ wild type, or Qβ mutant, antigen conjugate as described above) in a pharmaceutical composition will be less than or equal to about 2.5 M, 2.4 M, 2.3 M, 2.2 M, 2.1 M, 2 M, 1.9 M, 1.8 M, 1.7 M, 1.6 M, 1.5 M, 1.4 M, 1.3 M, 1.2 M, 1.1 M, 1 M, 0.9 M, 0.8 M, 0.7 M, 0.6 M, 0.5 M, 0.4 M, 0.3 M or 0.2 M. In some embodiments the concentration of the one or more vaccine compositions (e.g., one or more Qβ wild type, or Qβ mutant, antigen conjugate as described above) will be less than or equal to about 0.10 mg/ml, 0.09 mg/ml, 0.08 mg/ml, 0.07 mg/ml, 0.06 mg/ml, 0.05 mg/ml, 0.04 mg/ml, 0.03 mg/ml or 0.02 mg/ml.

Actual dosage levels of the active ingredients in the compositions of the present invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular therapeutic agent in the formulation employed, or the ester, salt or amide thereof, the route of administration, the time of administration, the rate of excretion or metabolism of the particular therapeutic agent being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above.

If desired, the effective daily dose of the active compound may be administered as two, three, four, five, six or more sub-doses administered separately at appropriate intervals throughout the day, optionally, in unit dosage forms.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing

While the subject is being treated, the health of the patient may be monitored by measuring one or more of the relevant indices at predetermined times during a 24-hour period. All aspects of the treatment, including supplements, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments, for example, to the amount(s) of agent administered and to the time of administration may be made based on these reevaluations.

Treatment may be initiated with smaller dosages which are less than the optimum dose of the compound. Thereafter, the dosage may be increased by small increments until the optimum therapeutic effect is attained.

Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the one or more vaccine compositions (e.g., one or more Qβ wild type, or Qβ mutant, antigen conjugate as described above) described herein relative to the control. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the one or more vaccine compositions (e.g., one or more Qβ wild type, Qβ mutant, antigen conjugate as described above) described herein relative to control. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the one or more vaccine compositions (e.g., one or more Qβ wild type, or Qβ mutant, antigen conjugate as described above) described herein relative to control. Although compounds that exhibit toxic side effects may be used, care should be taken to design a delivery system that targets the compounds to the desired site in order to reduce side effects.

In some embodiments, the presently disclosed methods produce at least about a 10%, 15%, 20%, 25%, 30%, 35%, 40%, 45%, 50%, 55%, 60%, 65%, 70%, 75%, 80%, 85%, 90%, 95%, or even 100% inhibition of cancer cell growth in an assay.

In any of the above-described methods, the administering of the one or more vaccine compositions (e.g., one or more Qβ wild type, or Qβ mutant, antigen conjugate as described above) can result in at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy in a subject, compared to the solid malignancy before administration of the vaccine compositions.

In some embodiments, the therapeutically effective amount of one or more vaccine compositions (e.g., one or more Qβ wild type, or Qβ mutant, antigen conjugate as described above) is administered prophylactically to prevent a solid malignancy from forming in the subject.

In some embodiments, the subject is human. In other embodiments, the subject is non-human, such as a mammal.

The data obtained from the cell culture assays and animal studies may be used in formulating a range of dosage for use in humans. The dosage of any supplement, or alternatively of any components therein, lies preferably within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC₅₀ as determined in cell culture. Such information may be used to more accurately determine useful doses in humans. Levels in plasma may be measured, for example, by high performance liquid chromatography.

### EXEMPLIFICATION

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1: OB Salmonella Glycan Conjugate as a Potential Anti-Salmonella Vaccine

Salmonella is a Gram-negative bacterium, which can cause serious infections (salmonellosis) through ingestion of contaminated food or water. Salmonella encompasses >2500 serovars distinguished by serotype based on the structure of the O polysaccharide on cell surface and by clinical syndrome. Typhoid and paratyphoid serovars (S. typhi and S. paratyphi) cause invasive enteric fever whereas nontyphoidal Salmonella generally cause gastroenteritis for which invasive disease can occur as a consequence of immunological immaturity, suppression, or senescence. S. typhi causes typhoid fever that kills over 200,000 people every year mainly in developing countries especially in Southeast Asia, Latin America and Africa.2 In Asia, paratyphoid due to S. paratyphi A is also prevalent. In US, nontyphoidal Salmonella (NTS) is more common and frequently associated with multistate outbreaks, which is responsible for >1.2 million infections, tens of thousands of hospitalizations and hundreds of deaths in people every year. Among the different NTS serovars associated with clinical illness in US, S. enteritidis and S. typhimurium are most common. According to 2010 data from Centers for Disease Control and Prevention (CDC)'s Foodborne Diseases Active Surveillance Network, salmonellosis accounted for 54 percent of hospitalizations and 43 percent of deaths reported due to food poisoning. Worldwide, there are an estimated 94 million cases and 155,000 deaths each year. While the incidence of food borne diseases such as Shiga toxin-producing E. coli 0157 infections has decreased more than 50% over the past two decades, no progress has been made in Salmonella with 10% increase of salmonellosis during the same period.

Currently invasive salmonellosis is treated with antimicrobials. However, multidrug resistant strains of Salmonella have emerged,9-10 with CDC classifying drug resistant Salmonella with the threat level of serious, which requires “prompt and sustained action to ensure the problem does not grow”. Despite the frequency and severity of salmonellosis and public health efforts to control the disease incidence, currently, there are no vaccines available against Salmonella strains other than S. typhi. For S. typhi, the ViCPS vaccine is particular noteworthy, which uses the purified Vi capsule polysaccharide from S. typhi directly as the immunogen. 13 This vaccine offers protection with between 50% to 80% efficacy in the first year, but loses its protection after two years. There is an urgent need to develop vaccines for Salmonella, especially against strains other than S. typhi, such as S. enteritidis, S. typhimurium and S. paratyphi A.

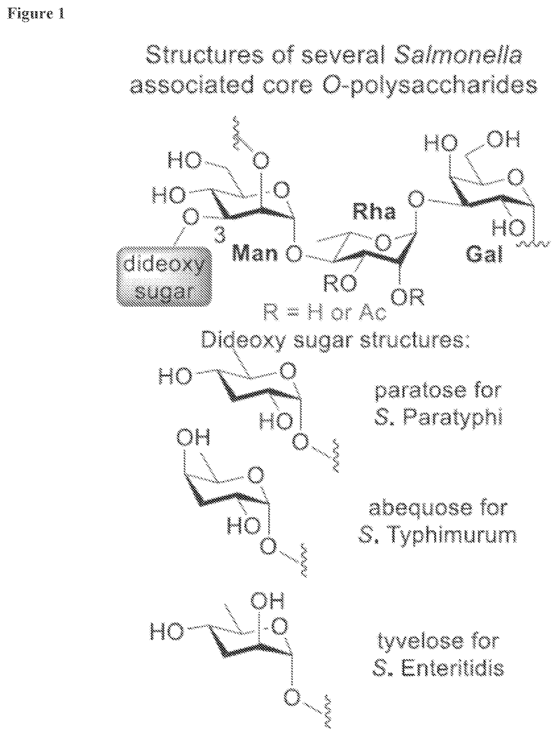

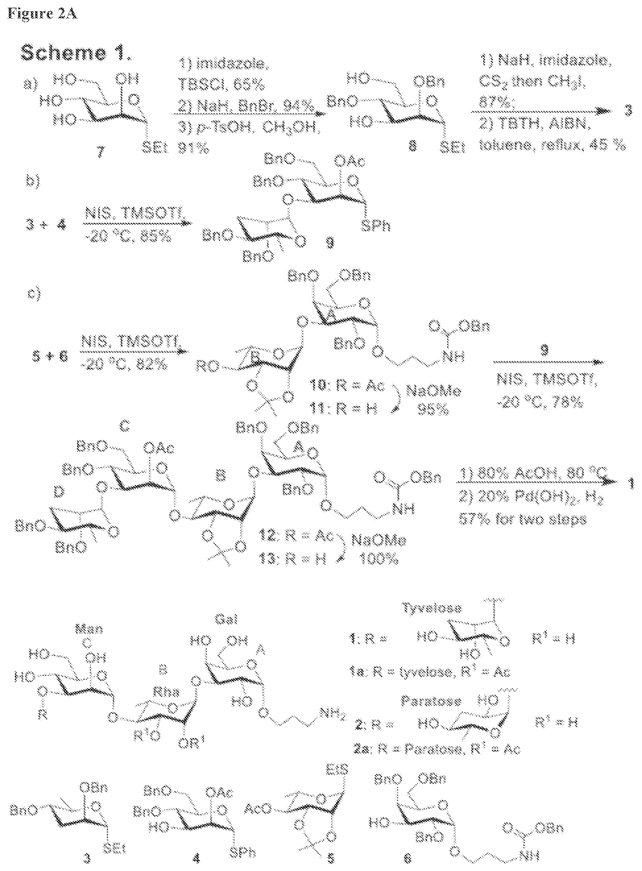

Vaccines targeting polysaccharides on cell surface of pathogenic bacteria have proven to be an effective strategy for protection from multiple bacterial pathogens, as exemplified by the ViCPS vaccine against S. typhi. For other Salmonella strains, while they lack Vi, there are characteristic surface polysaccharides. The cell surface core O-polysaccharides (COPS) of S. paratyphi, S. typhimurium, and S. enteritidis share a common backbone structure with repeating trisaccharides of α-D-mannose (Man)-1,4-α-L-rhamnose (Rha)-1,3-α-D-galactose (Gal)-1,2-. A dideoxy hexose is linked to the 3-OH of the Man and the structure of the dideoxy sugar differentiates the various serovars. S. paratyphi A contains paratose, S. typhimurium has abequose, while the dideoxy sugar is tyvelose on S. enteritidis. (See FIG. 1.) In some polysaccharides, the 6-OH of the backbone galactose can have an α-glucosyl unit attached. Furthermore, some of the 2- and 3-OH groups of the rhamnose are acetylated, resulting in further heterogeneities of natural glycans.

To date, all carbohydrate based anti-Salmonella vaccines utilize purified polysaccharides directly or as digestion products of polysaccharides derived from cultures of the corresponding serovar. These processes need to be strictly controlled to remove endotoxins and other harmful contaminants. Furthermore, the polysaccharides are a mixture with inhomogeneous lengths and structures, which can have batch-to-batch variability rendering it challenging to better understand the key structures of the protective epitopes. The possibility of using synthetic oligosaccharides with well-defined structures to develop vaccines against Salmonella is contemplated herein. These glycan antigens may be delivered using a powerful virus like particle (VLP) bacteriophage Qβ based carrier system to generate strong and long lasting humoral immune responses.

One of the major hurdles in developing glycan based anti-Salmonella vaccines is that carbohydrates are weakly immunogenic T cell independent antigens. When administered alone, they can only generate low titers of IgM antibodies, which typically have low affinity and only last for a short time. For an effective and durable vaccine, it is highly desirable that IgG antibodies can be elicited. In order to accomplish this, glycans can be attached to immunogenic protein carriers, which contain the necessary helper T (Th) cell epitopes to induce Th cell activation and antibody isotype switching from IgM to IgG. The loss of immune protection from ViCPS after two years is likely due to the fact that the ViCPS is administered without any Th epitopes.

Another critical parameter in carbohydrate-based vaccine design is how antigens are presented to B cells. This is because B cell activation and differentiation requires two signals: an antigen-specific first signal delivered via binding and cross-linking of cell surface B cell receptors (BCRs) and a second co-stimulatory cytokine signal from cognate Th cells. Arrays of haptens spaced by 5 to 10 nm have been shown to cross-link BCRs well and antigens displayed in a highly organized manner can lead to earlier B cell amplification for potent IgM and IgG responses. Antigen organization can also greatly influence B cell tolerance, with B cells unresponsive to poorly organized antigens while responding promptly to the same antigen presented in an orderly manner. Thus, a carrier capable of organized antigen display will be highly advantageous.

VLPs can be powerful platforms for antigen delivery. This is because in contrast to amorphous protein carriers such as bovine serum albumin (BSA), keyhole limpet hemocyanin (KLH) and tetanus toxoid (TT), VLPs are composed of self-assembled protein subunits giving them highly ordered structures. Repetitive B cell epitopes presented on the VLP surface can efficiently cross-link BCRs. Furthermore, VLPs can contain a large number of Th epitopes, which can synergize BCR cross-linking with stimulation by activated Th cells. Other advantages of VLPs include ease of genetic manipulation to engineer the particles for better immune potentiation, their high stabilities towards a variety of chemical modification conditions, speed and scale of production, and the ability to incorporate immuno-activating agents inside the capsid.

Recently, VLPs have been utilized as carriers of tumor associated carbohydrate antigens (TACAs). Similar to Salmonella associated glycans (SAGs), TACAs are T cell independent antigens. Out of the multiple types of VLPs investigated, bacteriophage Qβ is the most powerful in terms of carrier function. By displaying TACAs in an organized manner on Qβ, enhancement of antibody responses was observed even against a very weak TACA, i.e., monomeric Tn antigen, with anti-Tn IgG titers exceeding 250,000. In comparison, Tn-KLH conjugates elicited no anti-Tn IgG antibodies. Importantly, immunization with Qβ-Tn significantly protected mice from the development of an aggressive tumor, while Qβ alone did not provide any survival benefits suggesting the non-specific immune response due to Qβ itself was not sufficient.

For another tumor associated antigen, mucin-1 (MUC1), head-to-head comparison has been performed between Qβ-MUC1 and MUC1 conjugate with KLH, a gold standard utilized in multiple clinical trials of carbohydrate based anti-cancer vaccines. Consistent with the results from Tn studies, Qβ was superior as Qβ-MUC1 was able to induce three times more anti-MUC1 IgG antibodies, and was significantly more effective in reducing tumor load in mouse tumor models than the corresponding KLH-MUC1 construct.

Through the cancer vaccine studies, several valuable lessons were learned in applying Qβ for glycan based vaccine design. 1) High density antigen display is critical for high IgG antibody responses. Qβ construct bearing ~140 copies of MUC1 was able to elicit 4 times more anti-MUC1 IgG antibodies compared to the corresponding construct with 30 copies of MUC1 even though both groups of mice received the same amounts of total MUC1. The same density dependent phenomenon was observed in Tn vaccine studies; 2) Th cell activation is important as ablation of Th cells prior to immunization almost completely abolished anti-glycan IgG responses; 3) Covalent conjugation of antigen with Qβ carrier is critical, since an admixture of Qβ and MUC1 produced less than 3% of the anti-MUC1 antibodies compared to that induced by the covalent conjugated Qβ-MUC1; 4) The structure of the linker utilized for conjugation of antigens to Qβ is important. Qβ-Tn constructs formed using the copper catalyzed azide-alkyne cycloaddition click reaction were much less effective in inducing tumor binding antibodies vs those formed with a flexible alkyl amide linker. This was attributed to potential antigen competition from the rigid triazole ring and steric exclusion/epitopic suppression effects due to anti-triazole antibodies formed.

Despite the success in anti-cancer vaccine development, Qβ has not been systematically studied as a carrier for bacterium associated glycans. It was hypothesized that organized display of SAGs on Qβ can lead to strong and long-lasting anti-SAG IgG responses as potential anti-Salmonella vaccines.

### Innovations:

1) This studies disclosed herein are built upon integration of immunology with modern synthetic and bioconjugation chemistry.
2) VLP Qβ disclosed herein represent an exciting new carrier for anti-microbial vaccine development. Qβ-SAG conjugates can potentially generate high titers of anti-SAG IgG antibodies and immunological memory, two crucial factors in preventing Salmonella infections.
3) Novel Qβ carriers are disclosed herein, which are associated with reduced antibody generation against the carrier itself while retaining the ability to activate bacterium specific Th cells for induction of even higher levels of anti-SAG IgG antibodies vs wild type Qβ. These carriers can be a platform technology applicable to vaccine development for diseases beyond salmonellosis.
4) The availability of synthetic SAG oligosaccharides remove the reliance of pathogenic bacteria culture to isolate polysaccharides for vaccine constructs, thus improving batch to batch consistency and reducing the possibility of highly active bacterial contaminants in the vaccine. The well characterized synthetic glycan structures allow study of the minimum length and structures of protective SAG epitopes.
5) Targeting the common polysaccharide backbone of S. enteritidis, S. typhimurium and S. paratyphi A is a new direction in vaccine design enabled by the synthetic backbone glycan as it is not practically feasible to obtain these structures through enzymatic or chemical digestion. Backbone based vaccines can potentially be broad spectrum, imparting protection against multiple Salmonella serotypes of medical importance.

### Synthesis of Salmonella Associated Oligosaccharide Antigens.

Traditionally, studies of carbohydrate based anti-Salmonella vaccines have relied on poly-saccharides isolated from the pathogenic strains. The glycans obtained through this approach exist as an inhomogeneous mixture with variable lengths and structures. Furthermore, there are concerns regarding batch to batch variability and potential contaminants. Instead of relying on isolation from natural sources, synthetic routes are disclosed to produce structurally well-defined Salmonella associated glycans.

### Synthesis of Strain Specific Salmonella Tetrasaccharides:

Tetrasaccharides from S. enteritidis and S. paratyphi A.

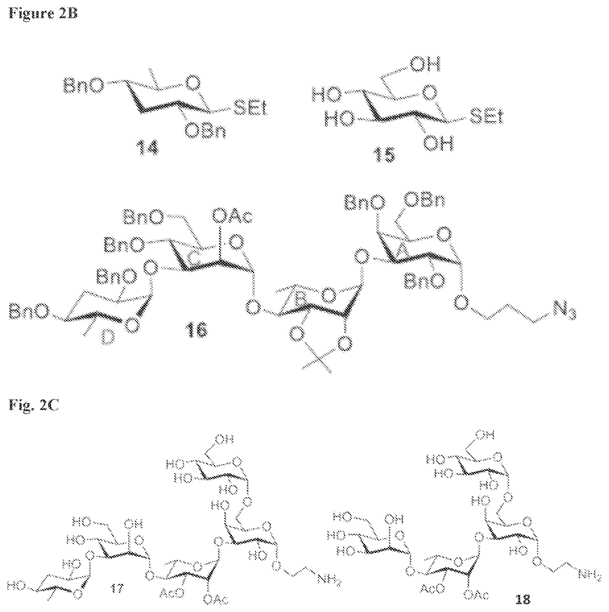

The first synthetic target was S. enteriditis tetrasaccharide 1, corresponding to one repeat unit with an amino propyl linker at the reducing end. Four monosaccharide building blocks 3-6 were designed, where benzyl (Bn) and acetonide groups were primarily used as protective groups as these electron-donating protective groups can potentially enhance building block reactivities in glycosylations. The key rare tyvelose donor 3 was efficiently prepared from the readily available thiomannoside which was selectively protected and then underwent Barton-McCombie deoxygenation leading to 3 (Scheme 1a; FIG. 2A). Building blocks 4-6 were prepared in a straightforward fashion. As deoxy sugars typically have higher anomeric reactivities than the fully oxygenated counterparts, selective activation of tyvelose 3 over the bifunctional mannoside 4 by N-iodosuccinimide (NIS, 1.2 eq.) and TMSOTf was achieved (Scheme 1b; FIG. 2A), furnishing α-tyvelosylated disaccharide 9 in 85% yield together with the minor β-anomer (~5%), which was separated by column chromatography. The new α-glycosidic linkage in compound 9 was confirmed by the coupling constant values of 13C-1 and H-1 [δ 99.3 (JC1/H1=167.8 Hz, C-1tyvelose), 85.8 (JC1/H1=167.8 Hz, C-1Man)]. Glycosylation of acceptor 6 by rhamnoside donor 5 produced disaccharide 10 as exclusive alpha anomer (Scheme 1c; FIG. 2A). Removal of the non-reducing end OAc from 10 followed by glycosylation by disaccharide donor 9 led to protected tetrasaccharide 12, which was subsequently deprotected generating tetrasaccharide 1 (75 mg). The structures of 1 including the stereochemistry of all glycosidic linkages were confirmed by NMR analysis. Alternatively, the 2-OAc in 12 was deprotected selectively (compound 13) for future chain elongation towards longer glycan. (See FIG. 2A.)

In order to synthesize S. paratyphi A tetrasaccharide 2, the paratose building block 14 was prepared from glucoside 15 through the same selective protection and Barton-McCombie deoxygenation applied for synthesis of tyvelose donor 3. The paratoside 14 and building blocks 4-6 were then utilized to synthesize the protected S. paratyphi A tetrasaccharide 16 in good overall yields following a similar chemoselective glycosylation approach for the preparation of 12. (See FIG. 2B.) Subsequent deprotection of 16 generated S. paratyphi A tetrasaccharide 2a (72 mg), which bears two OAcs in its rhamnoside unit for future investigation of the role of OAc in eliciting antibody responses. In addition, to better understand the roles of glycan structures in antibody recognition, penta-saccharide 17 bearing a glucoside on the reducing end Gal, and tetra-saccharide 18 without the paratose unit were also synthesized (90 mg each). (See FIG. 2C.)

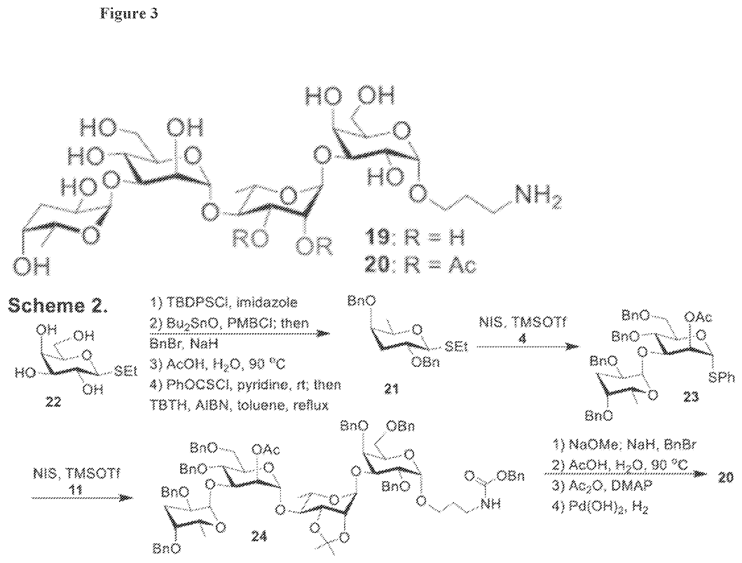

S. typhimurium is another major strain of Salmonella. In order to develop vaccines against S. typhimurium, S. typhimurium tetrasaccharides 19 and 20 are synthesized. (See FIG. 3.) Preparation of 19 and 20 will start from abequose donor 21 (Scheme 2), which in turn will be prepared by selective protection of galactoside 22 followed by Barton-McCombie deoxygenation. Chemoselective glycosylation of mannoside 4 by abequose 21 will lead to disaccharide 23. Analogous to synthesis of S. enteriditis and S. paratyphi A tetrasaccharides, the expected major anomer formed will be alpha, which will be confirmed by NMR. Coupling of disaccharide 23 with disaccharide 11 will produce tetrasaccharide 24, which will be deprotected producing S. typhimurium tetrasaccharide 20. Skipping the acetylation step in deprotection of 24 will yield tetrasaccharide 19.

Synthesis of the Strain Specific Octa- and Dodeca-Saccharides from S. enteritidis, S. paratyphi A, and S. typhimurium as Well as Common Backbone Oligosaccharides.

As naturally existing Salmonella O-polysaccharides consist mainly of letrasaccharide repeats, it is possible that oligosaccharides longer than one tetrasaccharide unit can lead to more potent anti-Salmonella antibody responses. Thus, octa- and dodeca-saccharides of the strain specific Salmonella glycans were synthesized containing two and three repeat units respectively. Targeting the polysaccharide backbone can potentially lead to broad spectrum vaccines against multiple strains of Salmonella. To enable this study, synthetic routes were developed to prepare backbone oligosaccharides.

Common Tri-, Hexa- and Nona-Saccharides of O-Polysaccharide Backbone have been Synthesized.

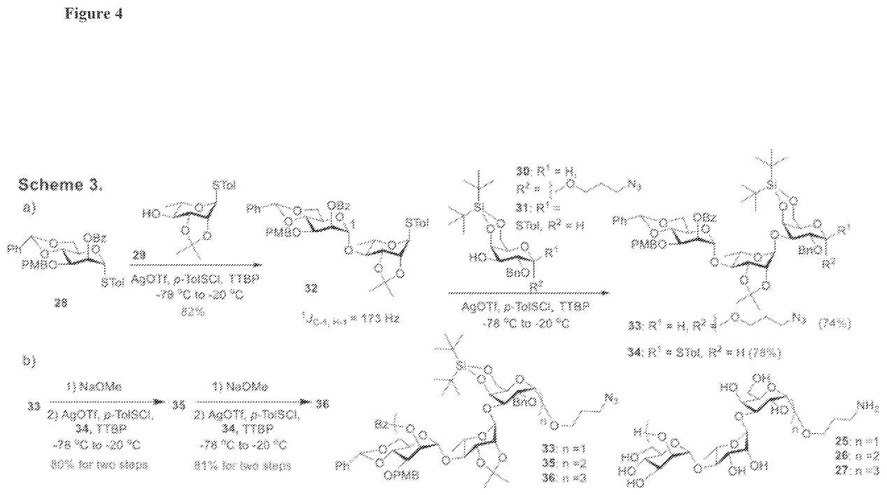

The common backbone of Salmonella consists of repeating trisaccharide of Man-Rha-Gal. The backbone oligosaccharides 25-27 were synthesized with one, two and three repeating trisaccharide units respectively (Scheme 3; FIG. 4). Monosaccharide building blocks 28-31 were established with protective group patterns for high alpha-selectivities in glycosylation. Pre-activation based glycosylation54-55 of mannoside donor 28 (1.2 eq.) with the bifunctional rhamnoside acceptor 29 (1 eq.) produced disaccharide 32 in 82% yield. This was followed by glycosylation of di-fbutyl silylene56 protected galactoside 31 leading to trisaccharide 34 in 78% yield (Scheme 3a). Similarly, trisaccharide 33 was synthesized in 74% yield using the galactoside 30 as the final acceptor. The Bz group of 33 was removed, and the resulting trisaccharide acceptor was glycosylated by donor 34 generating hexasaccharide 35 (80% yield) as the sole anomer isolated (Scheme 3b). The alpha configuration of the newly formed glycosidic linkage was confirmed by its 1JCH value of 173.5 Hz, which was presumably aided by the di-tbuyl silylene moiety. Repeating the Bz removal and glycosylation by trisaccharide donor 34 led to the fully protected nonasaccharide 36 (81% yield), which was successfully deprotected producing the Salmonella backbone nonasaccharide 27. The tri- and hexa-saccharide 25 and 26 were synthesized analogously (10 mg each) and fully characterized by NMR and MS.

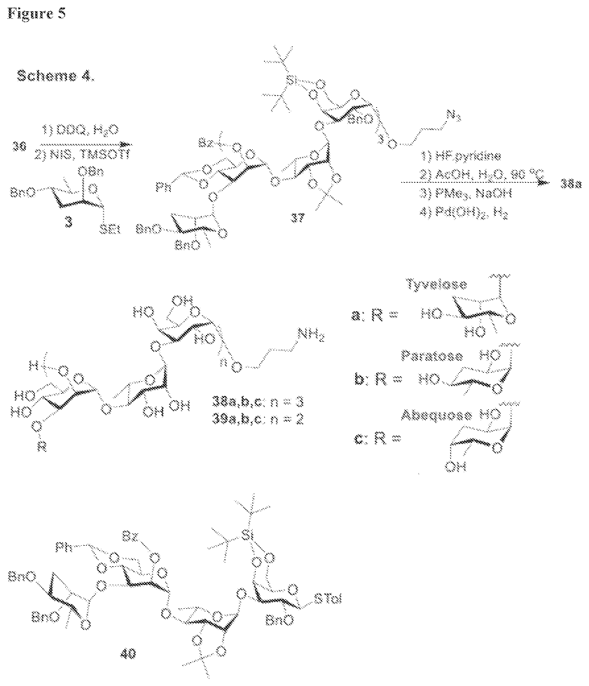

With the success in synthesis of common backbones 25-27, the most straightforward route to access the strain specific Salmonella glycans would be to convert these backbone glycans. As exemplified in Scheme 4 (FIG. 5), selective removal of the PMB groups from nonasaccharide 36 followed by glycosylation with >3 eq. of tyvelose donor 3 will generate the dodecasaccharide 37, which will be deprotected producing S. enteritidis dodecasaccharide 38a. Similarly, S. enteritidis octasaccharide 39a, S. paratyphi A octa- and dodeca-saccharides and S. typhimurium dodeca- and octa-saccharides 38b,c and 39b,c will be synthesized. This gives a divergent route, enabling rapid access of the array of SAG. The synthesis of all the target glycans outlined in aim 1 will be scaled up to 1 g each to support vaccine studies. As glycosylation proceeds with overall good yields without the need for large excess of building blocks, it is not expected significant difficulties in achieving this goal. It is possible that the triple glycosylation of 36 by 3 does not give high alpha selectivity for all three sites. If this is the case, tetrasaccharide module such as 40 will be synthesized bearing the deoxy sugar unit, which will then be utilized to assemble larger oligosaccharides.

### Development of Strain Specific Anti-Salmonella Vaccines.

SAGs by themselves are only weakly immunogenic, unable to elicit potent antibody responses when administered alone. Thus, it is critical that SAGs are covalently conjugated with an immunogenic carrier. Bacteriophage Qβ has been shown to be a powerful carrier for carbohydrate based anti-cancer vaccines. It was tested whether bacteriophage Qβ with SAGs can elicit powerful protective antibody responses as strain-specific anti-Salmonella vaccines.

Conjugation of SAG with Bacteriophage Qβ Carrier can be Carried Out Efficiently.

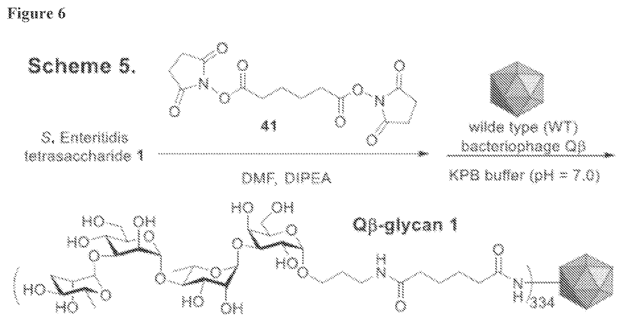

With the requisite strain-specific glycans synthesized, bio-conjugation to wild type (WT) Qβ was performed. The S. enteritidis tetrasaccharide 1 was functionalized with adipic acid di-N-hydroxysuccinimide (NHS) ester 41 (Scheme 5; FIG. 6), and then incubated with Qβ (5 eq of glycan per amine moiety on Qβ external surface; total 20 eq of glycan per Qβ monomer). The resulting Qβ-glycan 1 conjugate was purified. The excess of unconjugated glycan was recovered in near quantitative yield in its free carboxylic acid form, which could be converted back to the NHS ester and re-used for bioconjugation. LC-MS characterization of the Qβ-glycan conjugate showed that there were on average 334 copies of glycan per Qβ particle. Twenty mg of Qβ-glycan 1 conjugate was synthesized. Following a similar synthetic scheme, Qβ conjugate with S. paratyphi A (paratose) tetrasaccharide conjugate 2 was synthesized (on average 290 copies of glycan 2 per Qβ particle, 10 mg of the conjugate prepared). Glycans 1 and 2 were also conjugated with BSA to aid in immunological analyses.

Immunization with Qβ-Glycan 1 Elicited Powerful Anti-Glycan IgG Responses in Both Mice and Rabbits

The humoral responses against Qβ-glycan 1 and Qβ-glycan 2 were evaluated. These conjugates were injected subcutaneously into groups of 5 mice (day 0) at doses of 1 and 4 μg of glycans respectively followed by two booster injections on days 14 and 28. Control groups (5 mice each) received unconjugated Qβ at the same protein level.

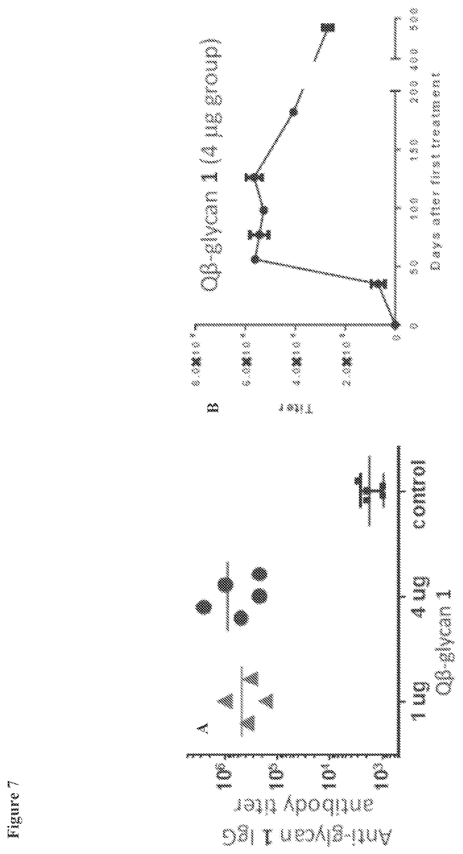

Enzyme linked immunosorbent assay (ELISA) analysis using BSA-glycan 1 as the coating antigen showed that high titers of IgG antibodies were induced by Qβ-glycan 1. The average IgG titers for the groups receiving 1 μg and 4 μg of glycan reached 487,000 and 980,000 ELISA units respectively on day 35 (FIG. 7A). In comparison, control groups immunized with Qβ only gave anti-glycan IgG titers below 5,000 ELISA units suggesting inclusion of glycan 1 in the vaccine was critical for high anti-glycan antibody responses.

For an effective vaccine, it is important that the immune responses induced to be long lasting. The antibody responses elicited by Qβ-glycan 1 were monitored over time (4 μg group data shown in FIG. 7B). The levels of anti-glycan 1 IgG antibodies reached its maximum value (6,000,000 ELISA units) 56 days after the first immunization, which was maintained for 120 days. Antibody titers gradually decreased, and at day 477, half of the peak IgG level remained. These results suggested that durable anti-glycan 1 IgG responses were induced by Qβ-glycan 1. No overt adverse effects were observed in immunized animals over the entire period highlighting the safety of the vaccine.

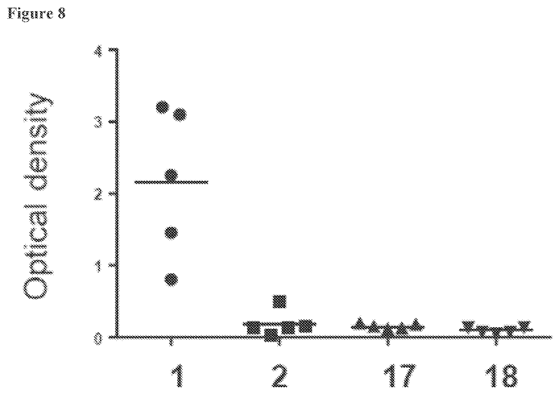

To establish the selectivity and specificity of antibody induced by Qβ-glycan 1, tetrasaccharide backbone 18, as well as S. paratyphi A tetrasaccharide 2 and pentasaccharide 17 were conjugated with BSA using linker 41 for ELISA. Analyses with these BSA glycan conjugates demonstrated that post-immune sera bound BSA-glycan 1 the strongest (FIG. 8), suggesting the high selectivities towards glycan 1 and critical role of the non-reducing end tyvelose in glycan 1 for antibody recognition.

In addition to mice, rabbits are another common animal model for immune evaluation. Two rabbits were immunized subcutaneously with Qβ-glycan 1 (4 μg glycan, biweekly injections). Sera were collected on days 0, 35, 49 and 56. ELISA analysis against BSA-glycan 1 showed that robust anti-glycan 1 IgG responses were induced, with IgG titers reaching 83,000,000 and 150,000,000 ELISA units by day 56 (Table 1), which were more than 6,000-fold higher than those from Qβ immunized control or pre-immunized rabbits. These results indicate that Qβ-glycan 1 is effective in multiple species.

TABLE 1
ELISA titers of anti-glycan 1 antibodies induced by Qβ-glycan
1 in rabbits. Robust anti-glycan 1 IgG antibodies were elicited
compared to control rabbits receiving Qβ only.

| | Day 35 | Day 49 | Day 56 |
|---|---|---|---|
| Rabbit #1 | 46,000,000 | 80,000,000 | 83,000,000 |
| w/Qβ-glycan 1 | | | |
| Rabbit #2 | 53,000,000 | 183,000,000 | 150,000,000 |
| w/Qβ-glycan 1 | | | |
| Control rabbit | <10,000 | <10,000 | <10,000 |
| w/Qβ only | | | |

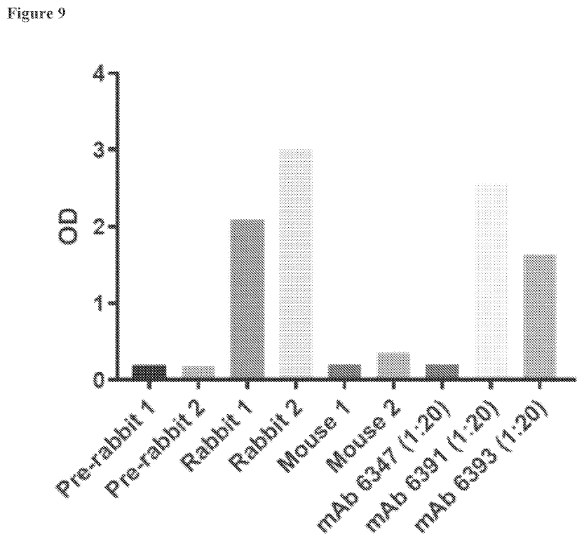

For successful vaccines, it is critical that antibodies generated by the vaccine construct are capable of recognizing the native polysaccharides. To test this, the homologous serotype native core O-polysaccharide (COPS) was isolated from S. enteritidis and immobilized in ELISA wells. Anti-sera from rabbits and mice were assessed for their ability to bind COPS. Three anti-Salmonella monoclonal antibodies (mAb 6347 against S. paratyphi A, mAb 6391 and 6393 against the core polysaccharide) were also added to the same ELISA plate for comparison. As shown in FIG. 9, mAb 6391 and 6393 recognized the COPS well while mAb 6347 did not. Sera from immunized rabbits bound the native COPS strongly. Higher COPS bindings were observed for rabbit sera compared to mouse sera. Similar species difference in anti-glycan antibody responses has been reported by Cummings group.

Antibodies Induced by Qβ-Glycan 1 Bound Strongly with S. enteritidis and Enhanced Macrophage Uptake of Bacteria.

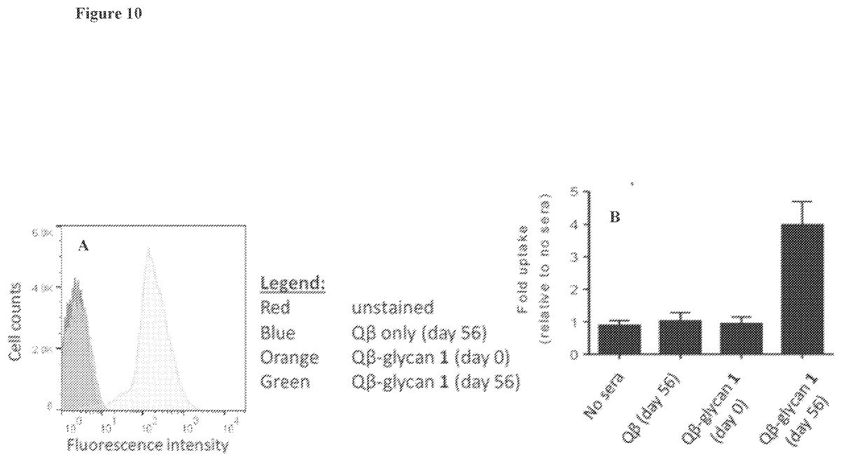

Whether the post immune sera could bind to the cognate antigen on intact bacterial cells was assessed using S. enteritidis strain R11 that was isolated from the blood of an infected toddler in Mali. Binding by post-immune sera from Qβ and Qβ-glycan 1 immunized rabbits with R11 was analyzed by flow cytometry. While negligible binding was seen for either the pre-immune sera or sera from rabbits immunized with the Qβ carrier alone, antisera from Qβ-glycan 1 immunized rabbits potently recognized R11 (FIG. 10A), which suggests the potential of Qβ-glycan 1 as an anti-S. enteritidis vaccine.

Opsonophagocytic uptake of antibody-bound bacteria by macrophages represents an important mechanism for antibody-mediated clearance of Salmonella. In order to determine whether post-immune sera have functional anti-Salmonella activity, uptake of S. enteritidis R11 by J774 mouse macrophages was assessed after incubation of the bacteria with pre-immune sera, anti-sera from Qβ or sera from Qβ-glycan 1 immunized rabbits. While the pre-immune and Qβ immunized rabbit sera did not cause significant bacterial uptake relative to media alone, antisera from Qβ-glycan 1 immunized rabbits enhanced macrophage opsonisation of bacteria by 400% (FIG. 10B).

Passive Transfer of Sera from Immunized Rabbits Protected Mice from Lethal Challenge by S. enteritidis.

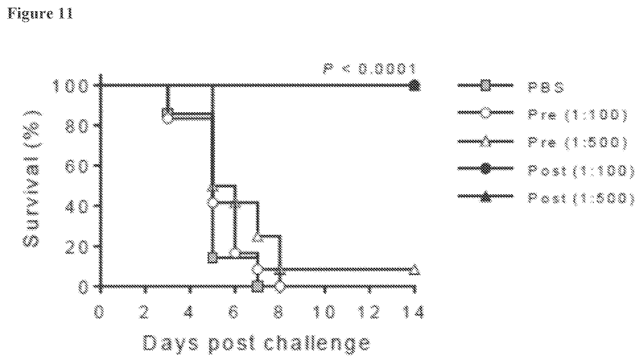

The protective efficacy of anti-sera against Salmonella infection was evaluated in vivo. Mice were passively administered PBS (n=7), different dilutions of pooled pre-immune (n=12/group) or vaccine-induced anti-sera (n=12/group) and then challenged intraperitoneally with a lethal dose (LD100) dose (1×10⁶ CFU) of R11. Whereas all mice in the PBS group succumbed to bacterial infection by day 7 and all but one out of the two groups of mice receiving pre-immune sera died from infection by day 8, excitingly, 100% of the mice receiving post-immune sera (1:100 or 1:500 dilution, 24 total) survived the bacterial challenge (FIG. 11).

Establishing the Optimal Length and Structure of S. enteritidis Glycan for Antibody Responses.

The preliminary immune evaluation focused on S. enteritidis tetrasaccharide antigen 1. As the native S. enteritidis COPS consist of a polymer of multiple repeats of the tetrasaccharide unit, it was hypothesized that longer glycans may mimic the conformational properties of native COPS better resulting in stronger antibody responses. Whether inclusion of octa- and dodeca-saccharides as antigens in the vaccine can result in superior antibodies in mice will be tested as the anti-Salmonella glycan antibody levels generated in mice were lower than those in rabbits.

The octa- and dodeca-saccharides 39a and 38a will be conjugated with Qβ through the bifunctional linker 41 in a similar manner as tetrasaccharide 1. Acetylation will be investigated by conjugating the acetylated tetrasaccharide 1a with Qβ. The resulting constructs will be thoroughly purified to remove free glycans. The glycan loading level will be quantified by LC-MS assay. If the loading levels are lower due to the larger sizes of the glycans, the bioconjugation reaction can be repeated to increase the loading to around 300 as in Qβ-glycan 1. The excess unconjugated glycans can be recovered and recycled.

Mice will be immunized with the new conjugates and the IgG antibody levels in the post-immune sera will be determined by ELISA against the immunizing antigen, as well as against the native COPS from S. enteritidis. Comparison of the antibody responses to those induced by Qβ-glycan 1 will help establish the impacts of antigen length and acetylation on recognition of COPS.

Vaccine dose is an important parameter to be optimized. In Qβ-Tn studies, it was observed that higher dose (20 μg) used in vaccination led to antisera with stronger binding of Tn expressing tumor cells than lower doses (1 and 4 μg). In preliminary studies, it was observed that mice immunized with Qβ glycan 1 at 4 μg glycan level trended towards higher mean IgG antibody levels compared to the 1 μg group (FIG. 7A). Higher dosages (8 and 40 μg antigen per injection) will be evaluated to establish the dose giving strongest glycan specific antibody titers. For the 40 μg dose, 10 mg Qβ-glycan conjugate will be sufficient for full immunization of 20 mice.

Adjuvants can significantly influence the immune responses. In preliminary studies, Freund's adjuvant was utilized. However, as Complete Freund's Adjuvant cannot be used in humans due to its toxicity, three adjuvants that are already used as part of human vaccines will be tested: unmethylated Cytosine-Guanine dinucleotides, Alum, and monophosphoryl lipid A (MPLA), which will be co-administered with Qβ-glycan conjugates. These adjuvants interact with Nod-like receptors (NLR) and Toll-like receptors (TLRs), which are key activators of the innate immune system. Previously, it was shown that Freund's adjuvant and MPLA induced similar levels of IgG antibodies. The adjuvant giving antibodies with the highest titers against COPS from S. enteritidis will be selected.

Mice will be monitored after immunization up to 2 years for the longevity of the IgG responses. According to preliminary data in FIG. 7B, it is possible that the anti-glycan IgG levels will remain high for the first 6 months and gradually decrease afterwards. If this is the case, another booster injection may be given at 1 year. This mimics the elder population in humans, which are a cohort more susceptible to Salmonella infections. The potential durability and boostability of the IgG responses will be attractive features of the vaccines disclosed herein.

Qβ-glycan immunization is well-tolerated. To date, despite the high IgG titers elicited, the only side effect observed due to immunization was ulceration at the injection site due to the toxicity of the complete Freund's adjuvant. Switching the adjuvant to MPLA completely removed this side effect in cancer vaccine studies. Any possible adverse effects will be monitored up to 2 years after immunization.

For constructs giving high antibody responses, the specificities of antibodies induced will be analyzed by screening the antisera on the microbial glycan microarray provided by the National Glycomics Center. This microarray contains over 300 representative carbohydrate structures from 36 species across 15 genera including many Salmonella strains. Based on preliminary screening (FIG. 8), it is expected the antibodies generated will be highly selective towards the S. enteritidis OPS glycan.

The binding ability of the antisera towards bacterial cells is tested by incubating post immune sera with S. enteritidis followed by flow cytometry analysis. For sera reacting strongly with the bacteria, the ability of the sera to clear the cells is determined by the macrophage opsonophagocytic assay (FIG. 10B) as well as complement mediated cytotoxicity assay (SBA). The construct giving IgG antibodies that exhibit strongest binding and cytotoxic effects towards S. enteritidis are identified. This provides critical information for the preferred epitope structure.

### Deeper Understanding of Immune Responses by Qβ-Glycan Conjugates.

To better understand the immune responses and immune memory induced by vaccination, memory B cells and long-lasting antibody secreting plasma B cells will be analyzed. Cells will be isolated from lymph nodes and spleens of mice immunized with Qβ only or Qβ-glycan 1. These cells will be subjected to flow cytometry analysis upon incubation with fluorescently labeled BSA-glycan 1 conjugate. Memory B cells populations will be quantified as cells that are SAG+IgG+B220+CD38+. If the frequency of these cells is too low for accurate quantification, the memory B cells can be differentiated into plasma cells by stimulation with lipopolysaccharide (LPS) for six days, and transferred to BSA-glycan 1 coated enzyme-linked immunospot assay (ELISPOT) plates to quantify the number of glycan specific IgG secreting B cells. Long-lasting glycan specific plasma B cells will also be evaluated by ELISPOT with cells from bone marrow. Qβ-glycan 1 immunization is expected to lead to a larger number of glycan specific memory B and long-lasting plasma B cells compared to controls.

Th cell activation is crucial for the generation of high affinity IgG antibodies and long term memory. To analyze Th activation by Qβ-glycan 1, lymphocytes from spleen or lymph nodes will be isolated from immunized as well as naïve mice, which will be followed by labeling with carboxyfluorescein diacetate succinimidyl ester (CFSE). Bone marrow dendritic cells (BMDCs) will be generated by culturing bone marrow cells from the tibias and femurs of naïve mice with DC-media containing granulocyte/macrophage colony-stimulating factor and interleukin-4. After stimulation with LPS, the mature BMDCs will be incubated with Qβ-glycan 1 and then added to CFSE labeled spleen cells. Proliferation of CD4+ T cells in the presence of Qβ-glycan 1 treated BMDCs will cause reduction of intracellular CFSE concentrations. Flow cytometry analysis of CFSE intensities of CD4+ cells will allow quantification of CD4+ T cells specific to the vaccine. A much greater percentage of CD4+ T cells are expected to be Qβ-glycan 1-specific in immunized mice compared to naive mice, which suggests CD4+ T cell activation induced by Qβ-glycan 1 vaccination.

Creating of Mutant Carriers Associated with Reduced Anti-Carrier Responses while Enhancing Anti-Glycan Antibody Levels.

A potential drawback in using immunogenic protein carriers is that anti-carrier antibody levels may drastically increase under prime-boost vaccination protocols. For example, a KLH-GD3 construct (1 prime and 2 boost injections) led to an anti-GD3 IgG titer of 400 with a 200× higher anti-KLH IgG titer (819,200). For Qβ-Tn, the difference was smaller at 12×: a mean IgG titer of 3,100,000 against Qβ vs. a mean anti-Tn IgG titer of 263,000.36 The endogenous epitopes from the carrier may compete with the desired epitope for the limited number of Th cells for cytokine signals, critical for B cell activation and IgG production. In addition, antibodies binding to the non-essential epitopes may sterically block the adjacent targeted epitope from being recognized by B cells or lead to immune complex removal. Thus, to help the immune system focus on the glycan epitopes in the conjugate, it is desirable to remove the non-essential carrier epitopes. Qβ mutant strategy to reduce anti-Qβ antibody responses and further enhance anti-glycan antibody production was investigated.

### Removing Qβ B-Cell Epitopes Lowered Anti-Qβ Titers and Increased Antibody Levels Against a Carbohydrate Antigen.

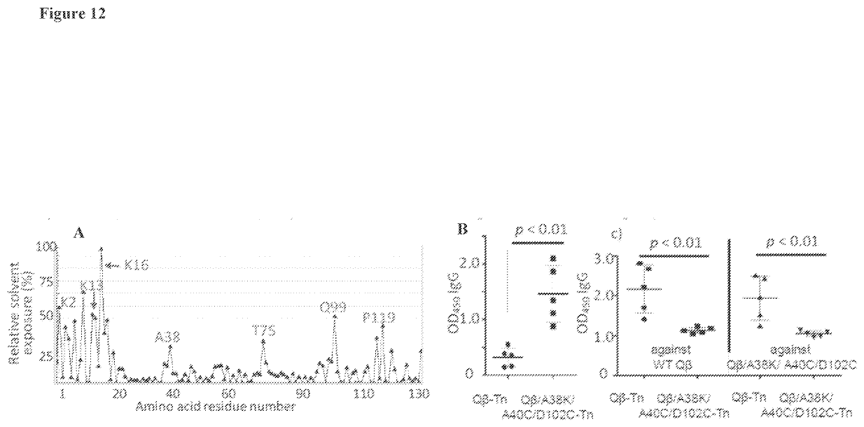

It was hypothesized that B cell epitopes likely reside in flexible and accessible loops on Qβ exterior as they need to bind to bulky B cell receptors on the surface of B cells. Based on the near atomic resolution crystal structure of Qβ, a triple mutant A38K/A40C/D102C was designed. A38 was selected since it is on the exterior and solvent exposed (FIG. 12A). The A40C and D102C mutations were introduced as these two residues are close in space in crystal structure, and can form a disulfide bond bridging two subunits to enhance capsid stability. The A38K/A40C/D102C mutant was expressed in E. coli and self-assembled to form particles in good yields (~20 mg/L) and similar sizes as wild type (WT) Qβ (28 nm diameter).

IgGs induced by a carbohydrate antigen Tn conjugated with Qβ/A38K/A40C/D102C (400 Tn per capsid) were evaluated. There were two notable findings: (1) the triple mutant elicited significantly higher anti-Tn IgG antibody levels (FIG. 12B); and (2) anti-carrier IgG titers against both WT Qβ and Qβ/A38K/A40C/D102C were much lower than those induced by WT Qβ-Tn (FIG. 12C). This suggests some inherent B-cell epitopes of Qβ was successfully removed without creating new dominant B-cell epitopes.

Following the rationale in generation of the Qβ/A38K/A40C/D102C triple mutant, the optimized glycan structure from above will be conjugated with Qβ/A38K/A40C/D102C triple mutant, and mice and rabbits will be immunized with the new constructs with the optimized dose and adjuvant. The IgG levels against COPS as well as those against the carrier will be determined by ELISA. In analogy to the results on Tn (FIG. 12B), the conjugate with the triple mutant carrier will produce superior IgG antibody responses compared to the corresponding WT-Qβ conjugate.

Besides the triple mutant, other exposed residues will be mutated including T75, Q99, A117, and P119 to amino acids such as lysine, alanine, and serine in order to further disrupt inherent B-cell epitopes of Qβ. Based on past experience, ~70% of Qβ mutants can self-assemble and form particles in good yields because selected mutation sites are not present at the interface between coat protein subunits. These new mutants (mQβ, m denotes mutant) will be conjugated with S. enteritidis glycan and immunize mice and rabbits. mQβ constructs giving highest anti-glycan IgG antibody responses, strongest COPS and bacterial binding will be identified as leads for further study.

### Establishment of a Heterologous Prime-Boost Strategy.

Qβ may have multiple major B cell epitopes. Therefore, effective Qβ mutations will be combined into one coat protein to test whether it can further reduce anti-Qβ IgG titers. However, with too many mutations, the coat protein may not assemble into particles. A heterologous prime-boost strategy using multiple mQβs containing different major B cell epitopes may be developed. One mQβ-glycan conjugate will be used to prime the immune system and a second mQβ-glycan conjugate for booster injections. In this strategy, as B-cell epitopes from various mQβ are different, glycan antigen is the only component common to all injections. Thus, anti-glycan immunity should benefit the most from booster injections. This heterologous prime-boost strategy should result in stronger anti-glycan antibody production and lower unwanted responses against the carrier.

Creating New Qβ Carrier Encapsulating Exogenous Salmonella Associated Th Cell Epitopes, which can Further Enhance Anti-Salmonella Antibody Levels and the Protective Efficacy.

The ability to induce high levels of anti-glycan IgG antibodies by Qβ-glycan conjugates suggests activation of Th cells leading to B-cell maturation and antibody isotype switch to IgG. However, as carbohydrates typically do not contain epitopes for Th cells, activated Th cells are most likely against the Qβ carrier. Furthermore, it has been shown that immunization with relevant Salmonella Th epitopes directly led to significant reduction of bacterial load in mice after Salmonella challenge. Thus, to enhance anti-Salmonella vaccine efficacy, it would be desirable to be include epitopes for Salmonella specific Th cells.

In order to activate Salmonella specific Th cells, Th cell epitope sequences from Salmonella need to be introduced into Qβ-glycan conjugates. While this can be accomplished by linking Th epitopic peptides to Qβ in the same manner as the glycans through linker 41, introduction of Th epitopes to the external surface of Qβ has several possible disadvantages: 1) it would reduce the number of sites available for glycan conjugation. As high density glycans on the surface of Qβ is important for high anti-glycan antibody levels, lowering the number of glycans on the capsid may adversely impact anti-glycan antibody levels; 2) Antibodies may be elicited against the exogenous Th epitopes, which can compete with the generation of anti-glycan antibodies.

To avoid potential complications for immobilizing Th epitopes on Qβ exterior, an RNA directed encapsidation strategy is contemplated herein to package the Salmonella Th epitopes in the Qβ interior. Due to steric hindrance, these Th epitopes in the interior will not be able to interact with B cells, thus minimizing induction of anti-Th antibodies. Furthermore, Qβ capsids can dissemble once taken up inside the cells, releasing the cargo. This is supported by the fact that RNA encapsulated inside Qβ activates intracellular Toll like receptor-9 pathway in antigen presenting cells after ingestion of Qβ.

The RNA directed encapsidation strategy is based on the understanding of the Qβ self-assembly process. Upon expression inside E. coli, the monomeric Qβ coat protein (CP) can bind with single-stranded RNA genome with high affinity between a RNA hairpin (hp) structure and positively charged residues of the coat protein. The RNA sequence serves as a template, and guides the assembly of the monomers into a 28 nm diameter nanoparticle.

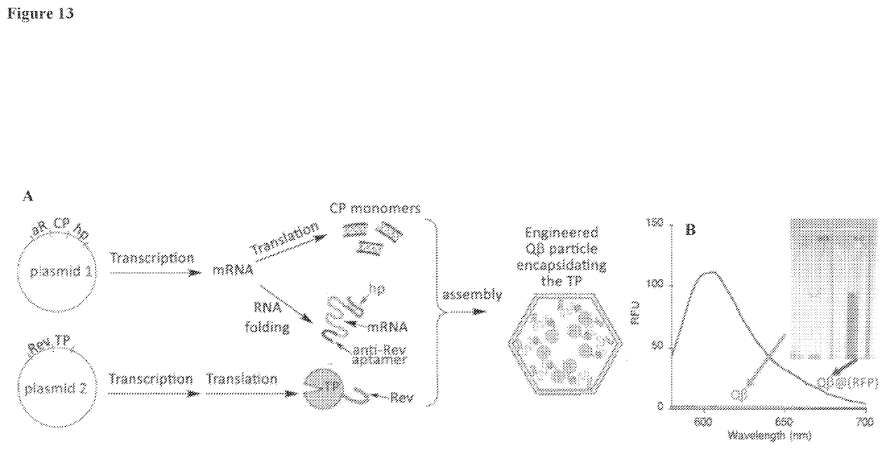

RNA-directed encapsidation is carried out by first introducing two binding domains to the mRNA sequence for Qβ coat protein on a plasmid (plasmid 1, FIG. 13A). An RNA aptamer (aR) capable of binding an arginine-rich peptide (Rev) is inserted just upstream of the ribosome binding site for the coat protein sequence. The sequence of the Qβ packaging hp is positioned immediately downstream of the stop codon. In parallel, the target protein (TP) sequence will be N-terminally tagged with the Rev peptide and inserted into plasmid 2. Transformation with both plasmids and expression in E. coli yields Qβ with the Rev-tagged target protein encapsidated by binding with the anti-Rev aptamer. Such Qβ is designated Qβ@ (protein) n with n indicating the average number of proteins inside a particle. This system was successfully utilized to encapsulate red fluorescence protein (RFP) in the interior of Qβ (FIG. 13B).

To apply the method for Salmonella vaccine development, a plasmid bearing the sequence corresponding to an N-terminus Rev tagged peptide containing Th epitope AAQYVAAHPGEVCPA from Salmonella alkyl hydroperoxide reductase subunit C (AhpC154-168) is constructed. Co-expressing this plasmid with the plasmid containing mRNA of the best mutant Qβ identified in aim 2.3 flanked by anti-Rev aptamer sequence and hp sequence in E. coli should encapsidate the Rev-tagged Th epitope inside mQβ, resulting in mQβ@(Th)n.

To evaluate the performance of mQβ@(Th)n, it will be functionalized with the optimized S. enteritidis glycan on the exterior. The resulting mQβ@(Th)n-glycan conjugate will be utilized to immunize mice using the optimized dose and adjuvant. Anti-COPS IgG titers and bacterium binding will be determined. In addition, the activation of AhpC154-168 specific Th cells will be determined using the CFSE assay outlined herein.

The protective efficacy of the vaccine construct with the new mQβ@(Th)n-glycan conjugate will be evaluated next. Mice will be fully immunized with the mQß@(Th)n-glycan construct. The comparison groups will be administered with WT Qβ-glycan and mQβ-glycan under identical conditions to decipher the impacts of mQβ and mQβ@(Th)n. Four weeks after the final booster, all mice will be injected with LD100 (1×106 CFU) of S. enteritidis R11. The number of mice surviving Salmonella challenge over time will be monitored to construct Kaplan-Meier survival curves as in FIG. 11.

In another experiment, two groups of mice immunized with mQβ@(Th)n-glycan and mQβ-glycan respectively will be kept for one year, and then challenged with S. Enteritidis. The recall responses from the mQβ@(Th)n-glycan immunized group will be assessed to determine relative protection to bacterial challenge.

To enhance Th activation, a new polypeptide may be designed to contain multiple repeats of Th epitopes AhpC154-168. In addition, other Th epitopes such as EutC243-257, STM1540262-276 and FliC429-443 can be introduced into the polypeptide as well. The ability to introduce multiple types of Salmonella Th epitopes as part of the vaccine is attractive for future translation into humans, as human leukocyte antigen (HLA) structures are diverse with varying Th epitope structure preferences. Multiple Th epitopes covering the major human HLA subtypes can be incorporated into Qβ for future translation.

As it is difficult to generate high anti-S. enteritidis glycan IgG responses in mice compared to rabbits, if the levels of antibody elicited in mice are not sufficient to be protective, mQβ@(Th)n-glycan conjugates in rabbits will be evaluated. Sera will be collected from immunized rabbits. Mice will be administered with serial dilutions of post-immune sera from immunized rabbits and then infected with lethal doses of S. enteritidis R11. The maximum dilution of sera giving 100% protection will be determined, which will indicate the strength of the protective responses. The passive transfer model is a useful alternative to the active immunization strategy.

Creating glycan based vaccines against S. typhimurium and S. paratyphi A. With the experience gained in S. enteritidis vaccine studies, the development of glycan based vaccines against S. typhimurium and S. paratyphi A using the best mQß@(Th)n platform is performed. Synthetic glycan antigens 2, 2a, 19, 20, 38b,c and 39b,c will be conjugated with mQβ@(Th)n using the same linker 41 as for S. enteritidis glycans.

Mice will be immunized with these constructs with three biweekly injections. Control groups of mice will receive mQβ@(Th)n admixed with the glycans without the covalent linkages. Blood will be collected pre-immunization as well as on days 7, 21, 35, 56 and 77 following the initial inoculation. In order to determine the anti-glycan antibody titers by ELISA, COPS will be purified from S. typhimurium and S. paratyphi A. The anti-COPS antibody titers and subtypes (IgG vs IgM, IgG subclasses) will be determined and compared at various time points to establish the kinetics of humoral responses and the necessity of booster injections.

The mQ@(Th)n-glycan conjugates will lead to superior anti-COPS IgG titers compared to control groups receiving mQβ mixed with glycans. As the epitope length preference and acetylation preference may be different from those of the S. enteritidis vaccine, the optimum length of the glycan epitope will be established by comparing binding to COPS from S. typhimurium and S. paratyphi A, binding to bacteria, and promotion of opsonization and complement mediated cytotoxicity by IgG antibodies induced by vaccines containing tetrasaccharide 2 or 19 vs the corresponding octa- and dodeca-saccharides 38b,c and 39b,c. The role of acetylation will be determined by comparing 2 vs 2a and 19 vs 20.

Based on the results from immunogenicity evaluation and in vitro analysis of COPS and bacterial binding/opsonization, two best constructs against S. typhimurium will be selected. Groups of mice will be immunized with the constructs with the adjuvant identified herein (3 biweekly doses). The control groups will receive admixture of mQβ@(Th)n and the glycan. The immunized mice will then be challenged with LD100 of S. typhimurium D65 (a clinical strain isolated from a Malian patient, 1×10⁵ CFU). The survival rate will be compared with control group receiving only PBS injection as well as those immunized with admixture of mQβ@(Th)n and the glycan with the same adjuvant. The construct giving the best protection will be identified.

For S. paratyphi A, currently there are no good animal models to evaluate vaccine-mediated protection. Thus, in vitro bacterial binding, complement mediated cytotoxicity and opsonization promoted by post-immune sera will be used as a surrogate for in vivo protection effects.

A novel mQβ(Th)n carrier with reduced anti-carrier response is disclosed herein, which upon conjugation with an optimized Salmonella glycan can activate Salmonella specific Th cells and elicit powerful strain specific protective immunity against lethal infections by S. typhimurum and S. enteritidis.

### Development of a Broad Spectrum Anti-Salmonella Vaccine

Besides the development of vaccines targeting specific strains, it would be attractive if a single vaccine construct could potentially protect against multiple common strains of Salmonella. As COPS from S. paratyphi A (serogroup A), S. typhimurum (serogroup B), and S. enteritidis (serogroup D) share a common trisaccharide backbone (Man-Rha-Gal), if strong functional antibody responses can be elicited against this structure, it is possible that a single vaccine can be effective against all three serogroups (serogroups A, B, D).

Evaluation of Qβ Conjugates with Salmonella Backbone Oligosaccharides.

The common backbone tri-, hexa- and nona-saccharides 25-27 were conjugated with wildtype Qβ using the bifunctional linker 41. The average loading levels were 246, 215 and 208 respectively for 25-27 based on LC-MS analysis. Mice (5 per group) and rabbits (2 per group) were immunized with these conjugates with three biweekly injections (4 μg of glycan per injection). One week after the final injection, anti-sera were collected and IgG antibody titers were analyzed against the immunizing antigen. Consistent with the strain specific glycan conjugates with Qβ, both mice and rabbits produced strong IgG responses (average IgG titers in mice are 1,500,000, while those for rabbits are 20,000,000).

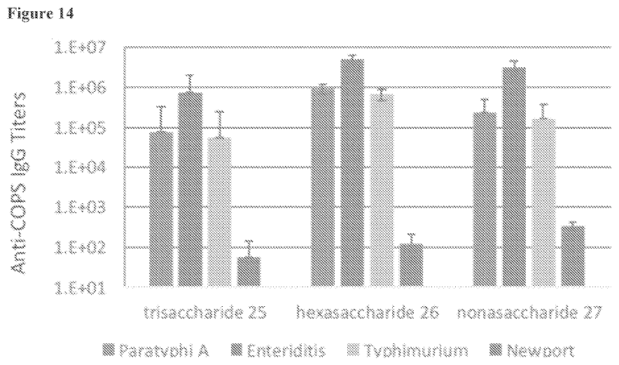

To analyze whether the antibodies produced can potentially recognize multiple strains of Salmonella, COPS from S. enteritidis, S. paratyphi A, S. typhimurum, and S. newport (serogroup C2-3) were purified and used as ELISA antigens. While S. enteritidis, S. paratyphi A, and S. typhimurum share the same COPS backbone, S. newport has a different COPS structure. The binding of the anti-sera with these four types of COPS were tested by ELISA (FIG. 14). Significantly higher levels of IgG antibodies were observed in the anti-sera recognizing the COPS from S. enteritidis, S. paratyphi A, and S. typhimurum, while those for S. newport were much lower (by ~500-fold). These results highlight the broad reactivity and selectivity of antibodies produced against the backbone structure.

Building on these results, it was hypothesized that conjugating backbone oligosaccharides with the new mQβ(Th)n carrier identified identified above would further enhance the strength of anti-Salmonella responses. The tri-, hexa- and nona-saccharides 25-27 will be conjugated with the mQβ(Th)n carrier.

Both mice and rabbits will be immunized with the new constructs and the adjuvant (3 biweekly injections). Control groups will receive mQβ(Th)n mixed with the glycans without covalent linkages and the adjuvant.

Post-immune sera will be collected from immunized animals and the abilities of the induced IgG antibodies to bind with COPS will be compared with those from the corresponding conjugate with WT Qβ as well as the control groups of mQβ(Th)n mixed with the glycans without covalent linkages. In preliminary studies, it was observed that while the conjugate of trisaccharide 25 can already generate strong IgG responses targeting COPS, the hexasaccharide antigen 26 elicited highest titers of IgG antibodies for COPS binding (FIG. 14). With the new carrier, the antigen length preference will be established in a similar fashion. The antigen giving the strongest responses will be evaluated further in challenge studies.

Clinical strains of S. enteritidis, S. typhimurum and S. paratyphi A will be cultured in addition to S. newport as a control. The binding of these bacteria by post-immune sera will be measured by flow cytometry. The COPS binding results suggest that the anti-sera will recognize S. enteritidis, S. typhimurum and S. paratyphi A strains strongly, while binding little to S. newport. Functional assays of antisera on the bacteria will be analyzed through the opsonic assays in the presence of macrophages (FIG. 10B) as well as the complement mediated cytotoxicity assay. The strong binding of anti-sera with all three strains (S. enteritidis, S. typhimurum and S. paratyphi A) will significantly enhance their uptake and killing in vitro, while the effects on S. newport strain should not be significantly different compared to control sera.

With the abilities of anti-sera against the S. enteritidis, S. typhimurum and S. paratyphi A strains established, in vivo protection studies will be performed. A group of mice will be immunized with the mQβ(Th)n-glycan construct and the adjuvant with the control group receiving admixture of mQβ and glycan. The immunized mice will be divided into two groups and be challenged with lethal doses of S. typhimurium D65 or S. enteritidis R11 respectively.

### Statistical Analysis and Ensuring Robust and Unbiased Results.

Log transformation of antibody titers and mean fluorescence intensities of cells due to antibody binding will permit examination of the multiplicative effects of new vaccine constructs. At a given dose level, ANOVA models followed by step-down Dunnett tests for pairwise treatment will control familywise Type I error rates for multiple testing while maximizing statistical power. Log-rank tests will be used to compare survival between groups. More advanced analyses will be performed in consultation with MSU's Center for Statistical Training and Consulting if necessary.

To evaluate immune responses, each sample will be tested on 3 separate occasions (biological replicates) to ensure reproducible results. The treatment regime will be blinded to researchers performing the measurements.

### Sex as a Biological Variable.

IgG responses induced by Qβ constructs from male and female mice were compared. The two sexes gave comparable levels of anti-glycan IgGs. Several other constructs will be tested similarly. If no significant differences appear, both sexes will be used in a cohort.

Currently there are no vaccines available against the major Salmonella strains, i.e., S. enteritidis, S. typhimurium and S. paratyphi A. By combining synthetic carbohydrate chemistry and Qβ engineering, developing effective carbohydrate based anti-Salmonella vaccines would fill this gap. Qβ conjugates with Salmonella associated glycans will be assessed for the ability to elicit powerful and enduring anti-Salmonella immunity, and protect immunized subjects from lethal challenges by Salmonella; laying the foundation to understand the various factors impacting anti-Salmonella glycan immune responses and provide an exciting new direction in salmonellosis prevention.

Common abbreviations used: BCR (B cell receptor); BSA (bovine serum albumin); CFSE (carboxyfluorescein diacetate succinimidyl ester); COPS (core O-polysaccharides); CP (coat protein), ELISA (enzyme linked immunosorbent assay); ELISPOT (enzyme-linked immunospot); hp (hairpin); KLH (keyhole limpet hemocyanin); LD100 (lethal dose); MPLA (monophosphoryl lipid A); SAG (Salmonella associated glycan); TACA (tumor associated carbohydrate antigen); Th (helper T cells); TLR (Toll like receptor); VLP (virus like particle); WT (wildtype)

### INCORPORATION BY REFERENCE

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

### EQUIVALENTS

Although the foregoing subject matter has been described in some detail by way of illustration and example for purposes of clarity of understanding, it will be understood by those skilled in the art that certain changes and modifications can be practiced within the scope of the appended claims.

What is claimed is:

1. A vaccine composition comprising a Salmonella antigen conjugated to a capsid, wherein said capsid comprises at least one non-natural mutation.

2. The vaccine composition of claim 1, wherein the capsid comprises at least one, two, three, four, five, six, seven, eight, nine, ten, eleven, twelve, thirteen, fourteen, fifteen, sixteen, seventeen, eighteen, nineteen, or twenty mutations.

3. The vaccine composition of claim 1, wherein the at least one non-natural mutation is a disulfide bond mutation.

4. The vaccine composition of claim 1, wherein the Salmonella antigen is derived from a surface glycan of Salmonella.

5. The vaccine composition of claim 4, wherein the Salmonella antigen is a polysaccharide.

6. The vaccine composition of claim 5, wherein the Salmonella antigen comprises tri-, tetra-, penta-, hexa-, hepta-, octa-, non, or dodeca-saccharides.

7. The vaccine composition of claim 4, wherein the Salmonella antigen comprises tri-, hexa-, or nona-saccharides of Salmonella O-polysaccharide backbone.

8. The vaccine composition of claim 7, wherein the Salmonella antigen comprises the trisaccharide of Man-Rha-Gal.

9. The vaccine composition of claim 1, wherein the capsid is a bacteriophage capsid.

10. The vaccine composition of claim 9, wherein the bacteriophage is selected from the group consisting of (a): bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (j) bacteriophage f2; (k) bacteriophage PP7; (l) bacteriophage AP205; and (m) bacteriophage P22.

11. The vaccine composition of claim 10, wherein the bacteriophage is bacteriophage Qβ.

12. The vaccine composition of claim 2, wherein the mutation comprises at least one mutation selected from N10K, A38K, A40C, A40S, T75K, D102C, D102S, or A117K, or combination thereof.

13. The vaccine composition of claim 12, wherein the mutation comprises A38K.

14. The vaccine composition of claim 2, wherein said capsid comprises at least two mutations selected from A40C/D102C, A40S/D102S, or A43C/Q98C.

15. The vaccine composition of claim 2, wherein said capsid comprises at least three mutations selected from A40C/D102C/K13R or A38K/A40C/D102C.

16. A method of preventing or treating a Salmonella infection (salmonellosis) in a subject, the method comprising administering to the subject a vaccine composition of claim 1.

17. A method of preventing or treating gastroenteritis, typhoid fever, and/or paratyphoid fever, the method comprising administering to the subject a vaccine composition of claim 1.

18. The method of claim 17, wherein, the gastroenteritis is chronic or acute.

19. The method of any one of claims 16-18, wherein the vaccine composition is administered systematically.

20. The method of claim 19, wherein the systematic administration is selected from the group consisting of oral, intravenous, intradermal, intraperitoneal, subcutaneous, and intramuscular administration.

21. The method of claim 16, wherein the vaccine is directed against multiple strains of Salmonella, selected from S. enteritidis, S. paratyphi A, S. typhimurum, and S. newport.

22. The method of claim 7, wherein the the Salmonella antigen is derived from a O-polysaccharide backbone present in one or more of S. enteritidis, S. paratyphi A, S. typhimurum, or S. newport.
